(19) United States
(12) Patent Application Publication
(10) Pub. No.: US 2024/0139268 A1
(43) Pub. Date: May 2, 2024

(54) HYBRID HASHISH-BASED CONSUMER PRODUCTS

(71) Applicant: Hexo Operations Inc., Gatineau (CA)

(72) Inventors: Todd Neault, Gatineau (CA); Renato Devien Durbano, Gatineau (CA); Sonia Patenaude, Gatineau (CA)

(73) Assignee: Hexo Operations Inc., Gatineau, QC (CA)

(21) Appl. No.: 18/269,455

(22) PCT Filed: Dec. 21, 2021
(86) PCT No.: PCT/CA2021/051863

§ 371 Date: Jun. 23, 2023

Related U.S. Application Data
(60) Provisional application No. 63/130,554, filed on Dec. 24, 2020.

(51) Int. Cl. A61K 36/185; A61K 9/16

(52) U.S. Cl. CPC A61K 36/185; A61K 9/1605; A61K 9/1694; A61K 2236/15

(57) ABSTRACT

The present disclosure relates to hybrid hashish products comprising a cohesive mass of agglomerated isolated Cannabis trichomes and particles of Cannabis plant biomass, and methods for manufacturing same. The method includes providing pre-treated raw materials for making the hybrid hashish product, the raw materials comprising pre-treated isolated Cannabis trichomes and pre-treated Cannabis plant biomass, comprising a Cannabis oil layer on at least a portion of a respective surface thereof; and mixing the raw materials under conditions sufficient to obtain a cohesive mass of agglomerated isolated Cannabis trichomes and particles of the Cannabis plant biomass.

### CROSS-REFERENCE TO RELATED APPLICATION

The present application claims the benefit of U.S. provisional patent application Ser. No. 63/130,554 filed on Dec. 24, 2020. The contents of the above-referenced document are incorporated herein by reference in their entirety.

### TECHNICAL FIELD

This application generally relates to the field of hybrid hashish-based consumer products and methods of manufacturing and using such products.

### BACKGROUND

Hashish (or hash) is a concentrated derivative of the dried resin glands, known as trichomes, of mature and unpollinated female Cannabis plants. Hash contains the same active ingredients as marijuana—including cannabinoids such as tetrahydrocannabinol and others—although at higher concentrations than the un-sifted buds or leaves from which dried marijuana is made, which is tantamount to higher potency. The trichomes may be removed from the plant material by mechanical means.

Separated trichomes have a powder appearance (referred to as “kief”) and are pressed to obtain blocks of hash, the color and pliability of which can vary widely based on the source material, the extraction method, and the production conditions. For example, dry-sift pressed hashish is usually solid, whereas water-purified hashish—often called bubble hashish—is often a paste-like substance with varying hardness and pliability. The color of a hashish product is most commonly light to dark brown, but can also vary from transparent to yellow, tan, black, or red.

Hand or mechanical presses are often used to produce hash products. However, hand presses are too small and inefficient for commercial volume production, while mechanical presses lead to variability of the finished hash product and an inconsistent product batch-over-batch. Furthermore, obtaining the desirable pliability and hardness requires a significant amount of “art” that is hardly reproduceable and the skills of the individual play a key role in defining the quality of the finished product—characteristics that are undesirable when designing and implementing industrial scale procedures.

Current methods of producing hash cannot ensure thorough mixing of the hashish components, and thus cannot ensure uniform and homogeneous distribution of hashish components. This leads to a hashish product with uneven and unpredictable distribution of cannabinoids throughout a unit of product, or across batches of product, which leads to an inconsistent user experience. Additionally, difficulties in thoroughly mixing hashish components can lead to a lack of uniform texture, consistency, and color in the hashish product, which can be off-putting to a user and can signal other inconsistencies in the product. The challenge of consistently and homogeneously distributing components within a product unit and across batches has limited the development of hash within the legal Cannabis industry.

Hashish manufacturers additionally face commercialization challenges when attempting to meet various local regulations that often limit the potency of hashish products. This is particularly challenging when manufacturing hashish with various Cannabis plant strains and/or with strains having high cannabinoid content, beyond the usual 10 wt. % to 20 wt. %, which limits flexibility in terms of possible line of hashish products that can be manufactured for given jurisdictions.

Considering the above, it would be highly desirable to be provided with a hash product, system or method that would at least partially alleviate the disadvantages of the existing technologies.

### SUMMARY

This Summary is provided to introduce a selection of concepts in a simplified form that are further described below in the Detailed Description. This Summary is not intended to identify key aspects or essential aspects of the claimed subject matter.

As embodied and broadly described herein, the present disclosure relates to a hybrid hashish product, comprising a cohesive mass of agglomerated isolated Cannabis trichomes and particles of Cannabis plant biomass.

Implementations of the hybrid hashish product can include one or more of the following features:
the particles of the Cannabis plant biomass are substantially homogeneously distributed throughout the cohesive mass.
a first content level of a detectable marker in a first discreet portion of the cohesive mass is within 15% of a second content level of the detectable marker, and wherein the second level is an average level of the detectable marker in the hashish product or in a batch of hashish products.
the first content level of the detectable marker is within 10%, or within 5% of the second content level of the detectable marker.
the detectable marker is a cannabinoid, a terpene, a flavonoid, chlorophyll, water, or any combination thereof.
the particles of Cannabis plant biomass represent up to about 50 wt. %, preferably from about 10 wt. % to about 40 wt. %, the percentage being expressed relative to a total weight of the cohesive mass.
the Cannabis plant biomass is selected from Cannabis flower, Cannabis buds, Cannabis trim, Cannabis sugar leaves, and a mixture thereof.
the particles of the Cannabis plant biomass have a size that passes through a sieve having a 10.0 mm pore size or less, preferably a 2.0 mm pore size.
the hybrid hashish product comprises a cannabinoid in an amount of from about 5 wt. % to about 90 wt. %, the percentage being expressed relative to a total weight of the cohesive mass.
the Cannabis plant biomass comprises a cannabinoid content of at least 5 wt. %.
the cannabinoid is one or more of tetrahydrocannabinol (THC), cannabidiol (CBD), cannabinol (CBN), cannabigerol (CBG), and any combinations thereof.
one or more additional components.
the one or more additional components comprises one or more cannabinoid(s), one or more terpene(s), one or more flavonoid(s), one or more flavoring agent(s), one or more coloring agent(s), or any combinations thereof.
the one or more additional components is substantially homogenously distributed throughout the cohesive mass.
the isolated Cannabis trichomes is dry-sift kief.
the isolated Cannabis trichomes are from one or more strain(s) of Cannabis plant.
the particles of Cannabis plant biomass are from one or more strain(s) of Cannabis plant that are different from the one or more strain(s) of Cannabis plant of the isolated Cannabis trichomes.
the particles of the Cannabis plant biomass are from one or more strain(s) of Cannabis plant that are the same as the one or more strain(s) of Cannabis plant of the isolated Cannabis trichomes.
the hybrid hashish product is configured to be loaded into a rolling medium or wrapper to form a smoking article.
the rolling medium or wrapper is made from a material selected from paper, hemp, cordia palm leaf, tendu leaf, flower petal, banana leaves, flax, sisal, rice straw, Cannabis leaves and esparto.
the hybrid hashish product has a length of from about 40 mm to about 300 mm.
the smoking article is a cigarette of hybrid hashish.
the hybrid hashish product is configured to be loaded into a smoking device.
the smoking device is a heat-not-burn device.
the hybrid hashish product has a length from about 40 mm to about 100 mm, preferably about 50 mm.
the hashish product is configured for being loaded into a heat-not-burn device mounting component.
the heat-not-burn device mounting component is a mounting tube or a cartridge.
the hashish product has a weight of from about 0.2 g to about 3.5 g.

As embodied and broadly described herein, the present disclosure also relates to a method for making a hybrid hashish product, comprising providing raw materials for making the hybrid hashish product, the raw materials comprising pre-treated isolated Cannabis trichomes and pre-treated Cannabis plant biomass, wherein the pre-treated isolated Cannabis trichomes and the pre-treated Cannabis plant biomass comprise a Cannabis oil layer on at least a portion of a respective surface thereof; mixing the raw materials under conditions sufficient to obtain a cohesive mass of agglomerated isolated Cannabis trichomes and particles of Cannabis plant biomass.

Implementations of the method can include one or more of the following features:
providing the raw materials comprises preheating the isolated Cannabis trichomes and Cannabis plant biomass under conditions sufficient to ooze Cannabis oil out therefrom and obtain the Cannabis oil layer on the at least portion of the respective surface thereof.
the preheating is performed in presence of water.
the water is incorporated in an amount of up to about 15 wt. %, preferably from about 5 wt. % to about 15 wt. %, more preferably from about 10 wt. % to about 15 wt. %, the percentage being expressed relative to a total weight of the raw materials.
the preheating is performed in a sealed container.
the preheating is performed at a temperature of from about 70° C. to about 130° C., preferably from about 80° C. to about 125° C., more preferably from about 120° C. to about 125° C.
the preheating is performed for a duration of from about 10 minutes to about 60 minutes, preferably from about 30 minutes to about 40 minutes.
the pre-treated Cannabis plant biomass provided in the first step is in the form of particles, wherein the particles have a size that passes through a sieve having a 10.0 mm pore size or less, preferably a 2.0 mm pore size.
the mixing causes pulverization of the Cannabis plant biomass into particles, wherein the particles have a size that passes through a sieve having a 10.0 mm pore size or less, preferably a 2.0 mm pore size.
the Cannabis plant biomass is selected from Cannabis flower, Cannabis buds, Cannabis trim, Cannabis sugar leaves, and a mixture thereof.
the Cannabis plant biomass being provided represents up to about 50 wt. %, preferably from about 10 wt. % to about 40 wt. %, the percentage being expressed relative to a total weight of the raw materials.
the mixing causes particles of the Cannabis plant biomass to be substantially homogeneously distributed throughout the cohesive mass.
the mixing causes a first content level of a detectable marker in a first discreet portion of the cohesive mass is within 15% of a second content level of the detectable marker, and wherein the second level is an average level of the detectable marker in the hashish product or in a batch of hashish products.
the first content level of the detectable marker is within 10%, or within 5% of the second content level of the detectable marker.
the particles of the Cannabis plant biomass are from one or more of Cannabis flowers, buds, sugar leaves, or trim.
the particles of Cannabis plant biomass represent up to about 50 wt. %, preferably from about 10 wt. % to about 40 wt. %, the percentage being expressed relative to a total weight of the cohesive mass.
the particles of the Cannabis plant biomass have a size of between about 0.1 mm and 10 mm.
the hybrid hashish product comprises a cannabinoid in an amount of from about 5 wt. % to about 90 wt. %, the percentage being expressed relative to a total weight of the cohesive mass.
the Cannabis plant biomass comprises a cannabinoid with a cannabinoid content of at least 5 wt. %.
the cannabinoid is one or more of tetrahydrocannabinol (THC), cannabidiol (CBD), cannabinol (CBN), cannabigerol (CBG), and any combinations thereof.
one or more additional components are incorporated into the hybrid hashish product.
the one or more additional components comprises one or more cannabinoid(s), one or more terpene(s), one or more flavonoid(s), water, one or more flavoring agent(s), one or more coloring agent(s), or any combinations thereof.
the one or more additional components are incorporated prior to, during, or after the first step.
the one or more additional components are incorporated during the mixing step.
the isolated Cannabis trichomes are dry-sift kief.
the isolated Cannabis trichomes are from one or more strain(s) of Cannabis plant.
the Cannabis plant biomass is from one or more strain(s) of Cannabis plant that are different from the one or more strain(s) of Cannabis plant of the isolated Cannabis trichomes.
the Cannabis plant biomass is from one or more strain(s) of Cannabis plant that are the same as the one or more strain(s) of Cannabis plant of the isolated Cannabis trichomes.
the mixing is mechanical.
the mixing includes applying compression and shear forces to the raw materials via a plurality of interpenetrate helicoidal surfaces within an elongated enclosure.
the elongated enclosure is a food grinder.
the elongated enclosure further includes at least one die orifice, wherein the at least portion of the cohesive mass is retrieved from the elongated enclosure through the at least one die orifice.
the plurality of interpenetrate helicoidal surfaces are on at least one screw, the method further comprising adjusting a rotational speed of the at least one screw to obtain the cohesive mass.
further includes cutting the hybrid hashish product into a pre-determined portion size.
further includes cutting the hybrid hashish product into a piece sized and shaped to be loaded into a rolling medium or wrapper to form a cigarette of hybrid hashish.
further includes loading the hybrid hashish product into the rolling medium or wrapper to make the cigarette of hybrid hashish.
further includes cutting the hybrid hashish product into a piece sized and shaped to be loaded into a smoking device.
the smoking device is a heat-not-burn device.
the hybrid hashish product has a length of from about 40 mm to about 100 mm, preferably about 50 mm.
the hashish product is configured to be loaded into a heat-not-burn device mounting component.
the heat-not-burn device mounting component is a mounting tube or a cartridge.
the mixing step further comprises heating while mixing the Cannabis trichomes and the Cannabis plant biomass.
the heating is performed to a temperature of from about 20° C. to about 100° C.
the mixing step further comprises applying a pressure to the isolated trichomes and Cannabis plant biomass of from about 5 psi to about 1500 psi.
the cohesive mass has an elongated body with a cross-section width of from about 1 mm to about 20 mm.

All features of exemplary embodiments which are described in this disclosure and are not mutually exclusive can be combined with one another. Elements of one embodiment can be utilized in the other embodiments without further mention. Other aspects and features will become apparent to those ordinarily skilled in the art upon review of the following description of specific embodiments in conjunction with the accompanying Figures.

### BRIEF DESCRIPTION OF THE DRAWINGS

A detailed description of specific exemplary embodiments is provided herein below with reference to the accompanying drawings in which:

FIG. 1A shows a non-limiting flowchart example of a process for making a hybrid hashish product in accordance with an embodiment of the present disclosure.

FIG. 1B to 1D show non-limiting examples of possible sub-steps of the process of FIG. 1A in accordance with embodiments of the present disclosure.

FIG. 2 shows a non-limiting example of optional post-processing steps for making the hybrid hashish product of FIG. 1A.

FIG. 3 shows various hybrid hashish products that can be produced with the optional post-processing sub-steps of FIG. 2.

FIGS. 4A and 4B show hybrid hashish products in accordance with embodiments of the present disclosure that are configured to be loaded into a heat-not-burn device.

FIGS. 5A-5C show various hybrid hashish products in accordance with embodiments of the present disclosure that are loaded into a rolling medium for smoking by a user.

FIGS. 6A-6C show various hybrid hashish products in accordance with embodiments of the present disclosure that are loaded into a rolling medium for smoking by a user.

FIG. 7 shows a partitioning example of a test sample.

FIG. 8A is a picture that shows pre-treated kief in a container.

FIG. 8B is a picture that shows hashish products obtained after 1 pass (I), 3 passes (II), 6 passes (III), and 9 passes of the pre-treated kief from FIG. 8A through a food grinder in accordance with an embodiment of the present disclosure.

FIG. 9A is a picture that shows raw materials containing kief and milled Cannabis flower in a container.

FIG. 9B is a picture that shows the product obtained after 4 passes of the raw materials from FIG. 9A through a food grinder.

FIG. 10A is a picture that shows the product obtained after 4 passes of raw material through a food grinder. The product was homogeneous but did not form a cohesive mass as adding slight finger pressure caused the product to crumble.

FIG. 10B is a picture that shows a hashish brick obtained after pressing the product from FIG. 10A into a brick. The hashish brick was homogeneous but did not form a cohesive mass as adding slight finger pressure caused the brick to crumble.

FIG. 11A is a picture that shows the product obtained after 1 pass of pre-treated raw material through a food grinder in accordance with an embodiment of the present disclosure. The product was homogeneous and formed a cohesive mass.

FIG. 11B is a picture that shows a hashish brick obtained after pressing the product from FIG. 11A into a brick. The hashish brick was homogeneous and formed a cohesive mass.

FIG. 12 is a pictures mosaic of a cohesiveness test performed on various hashish product samples.

In the drawings, exemplary embodiments are illustrated by way of example. It is to be expressly understood that the description and drawings are only for the purpose of illustrating certain embodiments and are an aid for understanding.

### DETAILED DESCRIPTION

The present technology is explained in greater detail below. This description is not intended to be a detailed catalog of all the different ways in which the technology may be implemented, or all the features that may be added to the instant technology. For example, features illustrated with respect to one embodiment may be incorporated into other embodiments, and features illustrated with respect to a particular embodiment may be deleted from that embodiment. In addition, numerous variations and additions to the various embodiments suggested herein will be apparent to those skilled in the art considering the instant disclosure which variations and additions do not depart from the present technology. Hence, the following description is intended to illustrate some embodiments of the technology, and not to exhaustively specify all permutations, combinations, and variations thereof.

The present inventors have developed a hybrid hashish product and industrial method of manufacturing same that addresses at least some of the above-identified disadvantages of the existing technologies.

The present inventors have developed a hybrid hashish product comprising a cohesive mass of agglomerated isolated Cannabis trichomes and particles of Cannabis plant biomass.

The present inventors have also developed methods of manufacturing such hybrid hashish product that includes mixing isolated Cannabis trichomes with Cannabis plant biomass under conditions sufficient to produce a cohesive mass. Advantageously, the isolated Cannabis trichomes and Cannabis plant biomass are pre-treated to comprise a Cannabis oil layer on at least a portion of a respective surface thereof.

For example, the herein described approach provides a technical effect in that the resulting hybrid hashish product may include a cannabinoid content and/or terpene profile, which can be modulated/controlled. For example, the cannabinoid content and/or terpene profile can be modulated/controlled with the addition of pre-determined amounts of Cannabis plant biomass having a different cannabinoid content and/or terpene profile than that one of the isolated Cannabis trichomes used to form the hybrid hashish product, thus resulting in an overall modulated/controlled cannabinoid content and/or terpene profile.

For example, the herein described approach provides a technical effect in that the resulting hybrid hashish product may contain a more complete terpene profile, and in the best-case scenario a full spectrum terpene profile, where the approach is less labor intensive compared to existing technologies. For example, while there have been some attempts to obtain hybrid hashish products by pressing kief with full spectrum Cannabis oil into a brick (e.g., hash hybrid from Indigenous Bloom, Canada), obtaining the full spectrum Cannabis oil adds a significant technical difficulty that increases overall manufacturing cost.

For example, the herein described approach provides a technical effect in that the resulting hybrid hashish product may deliver consistent amounts of cannabinoids, terpenes, flavonoids, and the like to the user during each use, thus providing a more consistently reproducible therapeutic or recreational user experience. This in turn can be advantageous in view of increasing consumer demands for a predictable therapeutic or recreational user experience.

These and other advantages may become apparent to the person of skill in view of the present disclosure.

### Hybrid Hashish Product

The hybrid hashish product of the present disclosure comprises a cohesive mass of agglomerated isolated Cannabis trichomes and particles of Cannabis plant biomass.

The product is a “hybrid” product in the sense that it contains isolated Cannabis trichomes, which is the traditional raw material used for making hashish, and it contains other Cannabis plant biomass particles, where the latter is typically considered undesirable materials when making hashish. Indeed, most of the Cannabis plant biomass in the flowers, leaves and other plant parts are actually extraneous to the majority of hashish manufacturing methods. Inclusion of Cannabis plant biomass in the herein described hybrid hashish product was counterintuitive. Indeed, prior art known to the inventors requires isolating trichomes from Cannabis plant biomass to concentrate the active ingredients (i.e., cannabinoids and terpenes) and to concentrate the available amounts of Cannabis oil required to ensure cohesiveness of hashish products. This is because cohesiveness of hashish products is typically obtained when the Cannabis oil oozes out from the isolated trichomes during the pressing steps (e.g., with industrial presses or with hand pressing)—adding Cannabis plant biomass to the isolated trichomes was thus expected to be detrimental in reducing the concentration of available Cannabis oil and thus reducing the cohesiveness of the hashish product. Surprisingly and unexpectedly, it was observed that the hybrid hashish product according to the present disclosure has a cohesive mass.

As used herein, the term “Cannabis trichomes” or “trichomes” generally refers to crystal-shaped outgrowths or appendages (also called resin glands) on Cannabis plants typically covering the leaves and buds. Trichomes produce hundreds of known cannabinoids, terpenes, and flavonoids that make Cannabis strains potent, unique, and effective.

As used herein, the term “isolated Cannabis trichomes” refers to trichomes that have been separated from Cannabis plant material using any method known in the art. The details of various methods for separating trichomes from the Cannabis plant are well-known in the art. For example, and without wishing to be limiting in any manner, the isolated Cannabis trichomes may be obtained, but without being limited to, mechanical separation of trichomes from the plant, such as sieving (also known as sifting) through a screen by hand or in motorized tumblers (see for example WO 2019/161509)—resulting in dry-sift kief, or by submerging the Cannabis plants in icy water (see for example US2020/0261824, which is herein incorporated by reference) and agitating to separate the trichomes from the plant and drying the trichomes. Because of inherent limitations to existing separation methods, some plant matter or other foreign matter can be present in isolated Cannabis trichomes in minimal residual amounts.

Isolated Cannabis trichomes is typically referred to as “kief” (also “keef” or “kif”) and has a powdery appearance. The kief is subsequently pressed or formed (e.g., in a ball) to obtain a hashish product. In preferred embodiments of the present disclosure, the isolated Cannabis trichomes is dry-sift kief.

The isolated Cannabis trichomes forming the hashish product of the present disclosure may originate from one or more than one strain of Cannabis plant. It is known amongst consumers of hashish and other Cannabis products that using isolated Cannabis trichomes produced from more than one strain of Cannabis plant allows a user to tune the psychoactive and/or entourage effect obtained by consuming the product. The mixing of Cannabis plant strains may also allow to adjust the final concentration of a component of the product, for example but not limited to the cannabinoid content. Additionally, use of more than one strain allows for improved product and waste management—important in commercial production.

As used herein, the term “Cannabis plant(s)”, encompasses wild type Cannabis (including but not limited to the species Cannabis sativa, Cannabis indica and Cannabis ruderalis) and also variants thereof, including Cannabis chemovars (or “strains”) that naturally contain different amounts of the individual cannabinoids. For example, some Cannabis strains have been bred to produce minimal levels of tetrahydrocannabinol (THC), the principal psychoactive constituent responsible for the high associated with it and other strains have been selectively bred to produce high levels of THC and other psychoactive cannabinoids.

### Cannabis Plant Biomass

The hybrid hashish product of the present disclosure comprises a cohesive mass of agglomerated isolated Cannabis trichomes and particles of Cannabis plant biomass.

The Cannabis plant biomass may be Cannabis flowers, Cannabis sugar leaves, Cannabis buds, Cannabis trim, or any combination thereof. “Flowers” or “buds” generally comprise the complex structures that tend to have higher concentrations of trichomes including, but not limited to colas, pistils, stigmas, and bracts (also called calyx). As used herein, the term “trim” generally refers to excess leaves a cultivator trims from their plants to fully maximize Cannabis plant's bloom and, in turn, get more desirable trichomes.

In some embodiments, the Cannabis plant biomass is provided in particle form during the manufacture of the hybrid hashish product. The Cannabis plant biomass particles can have been made by mechanical processes such as with an ordinary coarse pulveriser, capable of grinding or milling Cannabis plant biomass, and the like. There are no particular limitations on the pulverization step, and the average particle size of the pulverized Cannabis plant biomass can be in the range of several hundreds of micrometers to several millimeters.

In some embodiments, the Cannabis plant biomass is provided during the manufacture of the hybrid hashish product (as will be described further below) in a whole form (i.e., whole bud, sugar leaves, trim, or flower) or partially shredded. Optionally, the Cannabis plant biomass can be at least partially dried (cured). For example, during manufacture of the hybrid hashish product, the whole or partially shredded Cannabis plant biomass may be pulverized into particles and incorporated into the cohesive mass.

The particles of the Cannabis plant biomass in the hybrid hashish product may have an average particle size of several hundreds of micrometers to several millimeters. For example, the particles of the Cannabis plant biomass can have a size that passes through a sieve having a 10.0 mm pore size or less. For example, the average particle size may be of between about 0.1 mm and about 10.0 mm, or any value therebetween. For example, the average particle size may be about 0.1 mm, about 0.2 mm, about 0.3 mm, about 0.4 mm, 0.5 mm, about 0.6 mm, about 0.7 mm, about 0.8 mm, about 0.9 mm, about 1.0 mm, about 1.25 mm, about 1.5 mm, about 1.75 mm, about 2.0 mm, about 2.25 mm, about 2.5 mm, about 2.75 mm, about 3.0 mm, about 3.5 mm, about 4.0 mm, about 4.5 mm, about 5.0 mm, about 5.5 mm, or about 6.0 mm, about 6.5 mm, about 7.0 mm, about 7.5 mm, about 8.0 mm, about 8.5 mm, about 9.0 mm, or about 9.5 mm, or in a range of values defined by the aforementioned values. Preferably, the particles of the Cannabis plant biomass have a size that passes through a sieve having a 2.0 mm pore size.

For example, a desired range of particle sizes may be obtained with techniques known in the art, such as separation with sieving, electrostatic separation, and the like. For example, the reader may opt to use a sieve that conforms to one or more of ASTM E11, AASHTO T-27 & M-27, NIST, ISO 3310-1, ISO 565/3310-1 and BS410 specifications. For example, the following table 1 lists several sieves based on ASTM E11 and ISO 565/3310-1 from which the reader can select a suitable sieve based on the desired application.

TABLE 1
Sieve Size Comparison Table

| ASTM E11 | | ISO 565/3310-1 |
|---|---|---|
| Standard | Alternate | Size |

| | | | | | |
|---|---|---|---|---|---|
| 8.0 | mm | 5/16 | in | 8.0 | mm |

| | | | |
|---|---|---|---|
| — | — | 7.1 | mm |

| | | | | | |
|---|---|---|---|---|---|
| 6.7 | mm | 0.265 | in | 6.7 | mm |
| 6.3 | mm | ¼ | in | 6.3 | mm |

| | | | | |
|---|---|---|---|---|
| 5.6 | mm | No. 3½ | 5.6 | mm |

| | | | |
|---|---|---|---|
| — | — | 5.0 | mm |

| | | | | |
|---|---|---|---|---|
| 4.75 | mm | No. 4 | 4.75 | mm |

| | | | |
|---|---|---|---|
| — | — | 4.50 | mm |

| | | | | |
|---|---|---|---|---|
| 4.00 | mm | No. 5 | 4.00 | mm |
| 3.55 | mm | — | 3.55 | mm |
| 3.35 | mm | No. 6 | 3.35 | mm |
| 3.15 | mm | — | 3.15 | mm |
| 2.80 | mm | No. 7 | 2.80 | mm |

| | | | |
|---|---|---|---|
| — | — | 2.50 | mm |

| | | | | |
|---|---|---|---|---|
| 2.36 | mm | No. 8 | 2.36 | mm |
| 2.00 | mm | No. 10 | 2.00 | mm |

| | | | |
|---|---|---|---|
| — | — | 1.80 | mm |

| | | | | |
|---|---|---|---|---|
| 1.70 | mm | No. 12 | 1.70 | mm |

| | | | |
|---|---|---|---|
| — | — | 1.60 | mm |

| | | | | |
|---|---|---|---|---|
| 1.40 | mm | No. 14 | 1.40 | mm |

| | | | |
|---|---|---|---|
| — | — | 1.25 | mm |

| | | | | |
|---|---|---|---|---|
| 1.18 | mm | No. 16 | 1.18 | mm |

| | | | |
|---|---|---|---|
| — | — | 1.12 | mm |

| | | | | |
|---|---|---|---|---|
| 1.00 | mm | No. 18 | 1.00 | mm |
| 0.850 | mm | No. 20 | 0.850 | mm |

| | | | |
|---|---|---|---|
| — | — | 0.800 | mm |

| | | | | |
|---|---|---|---|---|
| 0.710 | mm | No. 25 | 0.710 | mm |

| | | | |
|---|---|---|---|
| — | — | 0.630 | mm |

| | | | | |
|---|---|---|---|---|
| 0.600 | mm | No. 30 | 0.600 | mm |

| | | | |
|---|---|---|---|
| — | — | 0.560 | mm |

| | | | | |
|---|---|---|---|---|
| 0.500 | mm | No. 35 | 0.500 | mm |

| | | | |
|---|---|---|---|
| — | — | 0.450 | mm |

| | | | | |
|---|---|---|---|---|
| 0.425 | mm | No. 40 | 0.425 | mm |

| | | | |
|---|---|---|---|
| — | — | 0.400 | mm |

For example, the following table 2 also lists several sieves from which the reader can select a suitable sieve based on the desired application.

TABLE 2

| U.S. MESH | MICRONS | INCHES | MILLIMETERS |
|---|---|---|---|
| 3 | 6730 | 0.2650 | 6.730 |
| 4 | 4760 | 0.1870 | 4.760 |
| 5 | 4000 | 0.1570 | 4.000 |
| 6 | 3360 | 0.1320 | 3.360 |
| 7 | 2830 | 0.1110 | 2.830 |
| 8 | 2380 | 0.0937 | 2.380 |
| 10 | 2000 | 0.0787 | 2.000 |
| 12 | 1680 | 0.0661 | 1.680 |
| 14 | 1410 | 0.0555 | 1.410 |
| 16 | 1190 | 0.0469 | 1.190 |
| 18 | 1000 | 0.0394 | 1.000 |
| 20 | 841 | 0.0331 | 0.841 |
| 25 | 707 | 0.0280 | 0.707 |
| 30 | 595 | 0.0232 | 0.595 |
| 35 | 500 | 0.0197 | 0.500 |
| 40 | 400 | 0.0165 | 0.400 |
| 45 | 354 | 0.0138 | 0.354 |
| 50 | 297 | 0.0117 | 0.297 |
| 60 | 250 | 0.0098 | 0.250 |
| 70 | 210 | 0.0083 | 0.210 |
| 80 | 177 | 0.0070 | 0.177 |
| 100 | 149 | 0.0059 | 0.149 |
| 120 | 125 | 0.0049 | 0.125 |
| 140 | 105 | 0.0041 | 0.105 |
| 170 | 88 | 0.0035 | 0.088 |
| 200 | 74 | 0.0029 | 0.074 |
| 230 | 63 | 0.0024 | 0.063 |
| 270 | 53 | 0.0021 | 0.053 |
| 325 | 44 | 0.0017 | 0.044 |
| 400 | 37 | 0.0015 | 0.037 |

The Cannabis plant biomass may have been harvested from a single Cannabis plant strain or from a plurality of Cannabis plant strains; the plurality of Cannabis plant strains may have the same, similar, or different respective cannabinoid compositions and concentrations. Furthermore, the Cannabis plant biomass may originate from Cannabis plants that are of the same or of different strains to that of the isolated Cannabis trichomes. The selection of Cannabis plant biomass may be based on a desired user experience, or may be driven by more practical considerations, such as inventory management considerations and/or cannabinoid content of the hybrid hashish product.

In some embodiments, the particles of the Cannabis plant biomass may represent from about 5 wt. % to about 50 wt. %, where the percentage is expressed relative to a total weight of the cohesive mass. For example, the particles of the Cannabis plant biomass may represent from about 5 wt. % to about 50 wt. %, or from about 10 wt. % to about 40 wt. %. For example, the particles of the Cannabis plant biomass may represent about 10 wt. %, about 15 wt. %, about 20 wt. %, about 25 wt. %, about 30 wt. %, about 35 wt. %, about 40 wt. %, about 45 wt. %, or about 50 wt. %, or any value therebetween, or in a range of values defined by the aforementioned values.

In some embodiments, the particles of Cannabis plant biomass are substantially homogeneously distributed throughout the cohesive mass.

By “substantially homogeneously distributed”, it is meant that particles of Cannabis plant biomass (and/or additional component, as described below) is substantially uniform throughout the cohesive mass, resulting in a hybrid hashish product with a substantially constant or uniform composition throughout each unit, across multiple hashish product units, or across multiple batches of hashish product units.

The level of homogeneity can be measured by detecting proportions of a detectable marker throughout any given sample, allowing for slight measured variations throughout the cohesive mass, e.g., <15% variations, or <10% variations; such slight variations within the cohesive mass will be deemed to be “substantially homogeneous” for the purposes of the present disclosure.

In some embodiments, the detectable marker can be the Cannabis plant biomass particles per se, the distribution of which being detectable using quantitative methods. For example, and without wishing to be limiting in any manner, the Cannabis plant biomass particles can be visually detected, for example by color.

In some embodiments, the detectable marker can be one or more detectable molecule. The one or more detectable molecule may be a component common to the Cannabis plant biomass and the isolated trichomes that is detectable using any suitable technique, such as for example Gas Chromatography/Mass Spectrometry (GC/MS), High Pressure Liquid Chromatography (HPLC), Gas Chromatography/Flame Ionization Detection (GC/FID), infrared spectrum (IR) spectroscopy, ultra-violet spectrum (UV) spectroscopy, Raman spectroscopy, and the like. Other techniques may involve measuring water activity, for example using a capacitive hygrometer (e.g., the Aqualab™ 4TE (Meter, USA)) using the chilled-mirror dew point technique, or may involve measuring water content, for example using a moisture analyzer (e.g., MA160 Infrared Moisture Analyzer (Sartorius AG, Germany) using the loss on drying technique (e.g., USP NF 731 Loss On Drying method).

Because the one or more detectable molecule is a component common to the Cannabis plant biomass particles and the isolated trichomes, measuring the level of distribution homogeneity thereof informs on the level of homogeneity of the hybrid hash product, i.e., the level of distribution of the Cannabis plant biomass particles in the hash product.

For example, the one or more detectable molecule may be one or more of the following: a cannabinoid, a terpene, a flavonoid, chlorophyll, water, or any combination thereof. Preferably, the detectable molecule is a cannabinoid.

For example, the detectable marker (e.g., Cannabis plant biomass particles and/or the detectable molecule) can be detected in at least 90 vol. %, or in at least 95 vol. %, or in at least 99 vol. %, or in 100 vol. % of the hashish product depending on specific implementations of the present disclosure.

Alternatively or additionally, the levels (or contents) of the detectable marker (e.g., Cannabis plant biomass particles and/or the detectable detectable molecule) in the hashish product of the present disclosure is substantially homogeneous, such that the hashish product includes a first content level of the detectable marker (e.g., Cannabis plant biomass particles and/or the detectable detectable molecule) in a first discreet portion of the cohesive mass that is within 15% of a second content level of the detectable marker (e.g., Cannabis plant biomass particles and/or the detectable detectable molecule). The second level is an average level of the detectable marker (e.g., Cannabis plant biomass particles and/or the detectable detectable molecule) in the hashish product or in a batch of hashish products. For example, the first content level of the detectable marker (e.g., Cannabis plant biomass particles and/or the detectable detectable molecule) and the second content level of the detectable marker (e.g., Cannabis plant biomass particles and/or the detectable detectable molecule) are present in a ratio first/second content levels of from 0.85 to 1.15. For example, the ratio first/second content levels is of about 0.90, or about 0.95, or about 1.00, or about 1.05, or about 1.10, or about 1.15 or any value therebetween, or in a range of values defined by the aforementioned values. For example, the first discreet portion can be a core portion of the hybrid hash product and the second discreet portion can be a peripheral portion of the hybrid hash product, where the content level of the detectable marker (e.g., Cannabis plant biomass particles and/or the detectable detectable molecule) and the ratio of first/second content levels can be determined based on the distribution test described later in this text.

### Cannabinoid Content

The hashish product of the present disclosure comprises one or more cannabinoid(s). The one or more cannabinoid(s) may be present endogenously in the isolated trichomes and/or Cannabis plant biomass used to make the hashish product or may be added in the form of an additional component (as described later in this text).

As used herein, the term “cannabinoid” generally refers to any chemical compound that acts upon a cannabinoid receptor such as CB₁ and CB₂. A cannabinoid may include endocannabinoids (produced naturally by humans and animals), phytocannabinoids (found in Cannabis and some other plants), and synthetic cannabinoids (manufactured artificially, for example cannabinoids produced in yeast, for example as described in WO WO2018/148848). Examples of suitable phytocannabinoids include, but are not limited to, cannabichromanon (CBCN), cannabichromene (CBC), cannabichromevarin (CBCV), cannabicitran (CBT), cannabicyclol (CBL), cannabicyclovarin (CBLV), cannabidiol (CBD, defined below), cannabidiolic acid (CBDA), cannabidiol monomethylether (CBDM), cannabidiol-C4 (CBD-C4), cannabidiorcol (CBD-C1), cannabidiphorol (CBDP), cannabidivarin (CBDV), cannabielsoin (CBE), cannabifuran (CBF), cannabigerol (CBG), cannabigerol monomethylether (CBGM), cannabigerolic acid (CBGA), cannabigerovarin (CBGV), cannabinodiol (CBND), cannabinodivarin (CBVD), cannabinol (CBN), cannabinol methylether (CBNM), cannabinol propyl variant (CBNV), cannabinol-C2 (CBN-C2), cannabinol-C4 (CBN-C4), cannabiorcol (CBN-C1), cannabiripsol (CBR), cannabitriol (CBO), cannabitriolvarin (CBTV), cannabivarin (CBV), dehydrocannabifuran (DCBF), Δ7-cis-iso tetrahydrocannabivarin, tetrahydrocannabinol (THC, defined below), Δ9-tetrahydrocannabionolic acid B (THCA-B), Δ9-tetrahydrocannabiorcol (THC-C1), tetrahydrocannabivarinic acid (THCVA), tetrahydrocannabivarin (THCV), ethoxy-cannabitriolvarin (CBTVE), trihydroxy-Δ9-tetrahydrocannabinol (triOH-THC), 10-ethoxy-9hydroxy-Δ6a-tetrahydrocannabinol, 8,9-dihydroxy-Δ6a-tetrahydrocannabinol, 10-oxo-Δ6a-tetrahydrocannabionol (OTHC), 3,4,5,6-tetrahydro-7-hydroxy-α-α-2-trimethyl-9-n-propyl-2, 6-methano-2H-1-benzoxocin-5-methanol (OH-iso-HHCV), Δ6a,10a-tetrahydrocannabinol (Δ6a,10a-THC), Δ8-tetrahydrocannabivarin (Δ8-THCV), Δ9-tetrahydrocannabiphorol (Δ9-THCP), Δ9-tetrahydrocannabutol (Δ9-THCB), derivatives of any thereof, and combinations thereof. Further examples of suitable cannabinoids are discussed in at least WO2017/190249 and U.S. Patent Application Pub. No. US2014/0271940, which are each incorporated by reference herein in their entirety.

Cannabidiol (CBD) means one or more of the following compounds: Δ2-cannabidiol, Δ5-cannabidiol (2-(6-isopropenyl-3-methyl-5-cyclohexen-1-yl)-5-pentyl-1,3-benzenediol); Δ4-cannabidiol (2-(6-isopropenyl-3-methyl-4-cyclohexen-1-yl)-5-pentyl-1,3-benzenediol); Δ3-cannabidiol (2-(6-isopropenyl-3-methyl-3-cyclohexen-1-yl)-5-pentyl-1,3-benzenediol); Δ3,7-cannabidiol (2-(6-isopropenyl-3-methylenecyclohex-1-yl)-5-pentyl-1,3-benzenediol); Δ2-cannabidiol (2-(6-isopropenyl-3-methyl-2-cyclohexen-1-yl)-5-pentyl-1,3-benzenediol); Δ1-cannabidiol (2-(6-isopropenyl-3-methyl-1-cyclohexen-1-yl)-5-pentyl-1,3-benzenediol); and Δ6-cannabidiol (2-(6-isopropenyl-3-methyl-6-cyclohexen-1-yl)-5-pentyl-1,3-benzenediol). In a preferred embodiment, and unless otherwise stated, CBD means Δ2-cannabidiol.

Tetrahydrocannabinol (THC) means one or more of the following compounds: Δ8-tetrahydrocannabinol (Δ8-THC), Δ8-tetrahydrocannabivarin (Δ8-THCV), Δ9-cis-tetrahydrocannabinol (cis-THC), Δ9-tetrahydrocannabinol (Δ9-THC), Δ9-tetrahydrocannabinolic acid A (THCA-A), Δ10-tetrahydrocannabinol (Δ10-THC), Δ9-tetrahydrocannabinol-C4 (THC-C4), Δ9-tetrahydrocannabinolic acid-C4 (THCA-C4), synhexyl (n-hexyl-Δ3THC). In a preferred embodiment, and unless otherwise stated, THC means one or more of the following compounds: Δ9-tetrahydrocannabinol and Δ8-tetrahydrocannabinol.

Examples of suitable synthetic cannabinoids include, but are not limited to, naphthoylindoles, naphthylmethylindoles, naphthoylpyrroles, naphthylmethylindenes, phenylacetylindoles, cyclohexylphenols, tetramethylcyclopropylindoles, adamantoylindoles, indazole carboxamides, quinolinyl esters, and combinations thereof.

A cannabinoid may be in an acid form or a non-acid form, the latter also being referred to as the decarboxylated form since the non-acid form can be generated by decarboxylating the acid form. Within the context of the present disclosure, where reference is made to a specific cannabinoid, the cannabinoid can be in its acid, its non-acid form, or be a mixture of both acid and non-acid forms.

The content in the acid form and the decarboxylated form of a specific cannabinoid can be determined using suitable methods known to the person skilled in the art, such as but not limited to Gas Chromatography/Mass Spectrometry (GC/MS), High Performance Liquid Chromatography (HPLC), Gas Chromatography/Flame Ionization Detection (GC/FID), Fourier transform infrared (FT-IR) spectroscopy, and the like. Various suitable methods are described, for example, in Formato et al. (—)-Cannabidiolic Acid, a Still Overlooked Bioactive Compound: An Introductory Review and Preliminary Research. Molecules. 2020 Jun. 5; 25(11):2638.

The hybrid hashish product of the present disclosure may contain one or more cannabinoid(s). The one or more cannabinoid(s) may originate from the Cannabis plant biomass, the isolated trichomes, from an additional component (as described below), or from a combination thereof. In some embodiments, the hybrid hashish product of the present disclosure may include one or more cannabinoid(s), such as THC, CBD, CBG, CBN, or any combinations thereof, in similar or different amounts.

In one embodiment, the hybrid hashish product of the present disclosure contains the one or more cannabinoid(s) in an amount (the “cannabinoid content”) sufficient for the user to experience a desired effect when consuming the product. For example, the hybrid hashish product may comprise from about 5 wt. % to about 90 wt. % cannabinoid, the percentage being expressed relative to a total weight of the cohesive mass, or any value therebetween, or in a range of values defined by any values therebetween. For example, the hybrid hashish product may comprise up to about 90 wt. %, up to about 80 wt. %, up to about 70 wt. %, up to about 60 wt. %, or up to about 50 wt. %, or up to about 40 wt. %, or up to about 30 wt. % or any value therebetween, or in a range of values defined by the aforementioned values.

### Additional Components

The hashish product according to the present disclosure may also comprise one or more additional components.

In some embodiments, the one or more additional component may be added to alter the characteristics of the hybrid hashish product, such as cannabinoid content, potency, entourage effect, odor, color, consistency, texture, pliability, and the like.

In some embodiments, the one or more additional component may be incorporated during the process to produce the hybrid hashish product, and similarly to the Cannabis plant biomass in particle form, may be substantially homogeneously distributed throughout the cohesive mass, or the one or more additional component may be substantially homogenously distributed along an external surface of the hybrid hashish product, for example as a coating.

The one or more additional component may be any suitable food grade and/or non-toxic composition or component known in the art. As will be recognized by those of skill in the art, the suitability of each type of additional component must be carefully considered prior to inclusion in the hybrid hashish product. For example, in applications where smoke/vapor produced by the hybrid hashish product is to be inhaled, suitable additional components may include, but are not limited to one or more cannabinoid, one or more terpene (also referred to herein as a “terpene blend”), one or more flavonoid, one or more flavouring agent, one or more non-toxic colouring agent, or any combination of any noted additional components.

In some embodiments, the one or more additional component in the hashish product of the present disclosure is a cannabinoid. For example, the cannabinoid may be extracted from any suitable source material including, but not limited to, Cannabis or hemp plant material (e.g., flowers, seeds, and trichomes), or may be manufactured artificially (for example cannabinoids produced in yeast, as described in WO WO2018/148848). Cannabinoids can be extracted from a Cannabis or hemp plant material according to any procedure known in the art. For example, and without wishing to be limiting, a “crude extract” containing a cannabinoid may be obtained by extraction from plant materials using for example aliphatic hydrocarbons (such as propane, butane), alcohols (such as ethanol), petroleum ether, naphtha, olive oil, carbon dioxide (including supercritical and subcritical CO₂), chloroform, or any combinations thereof. There are various terms used in the art to refer to Cannabis crude extracts, such as shatter, wax, to name a few. Optionally, the crude extract may then be “winterized”, that is, extracted with an organic solvent (such as ethanol) to remove lipids and waxes (to produce a “winterized extract”), as described for example in U.S. Pat. No. 7,700,368, US 2004/0049059, and US 2008/0167483, which are each herein incorporated by reference in their entirety. Optionally, the method for obtaining the cannabinoid may further include purification steps such as a distillation step to further purify, isolate or crystallize one or more cannabinoids, which is referred to in the art and herein as a “distillate”; US20160346339, which is incorporated herein by reference, describes a process for extracting cannabinoids from Cannabis plant material using solvent extraction followed by filtration, and evaporation of the solvent in a distiller to obtain a distillate. The distillate may be cut with one or more terpenes. The crude extract, the winterized extract or the distillate may be further purified, for example using chromatographic and other separation methods known in the art, to obtain an “isolate”. Cannabinoid extracts may also be obtained using solvent-less extraction methods; for example, Cannabis plant material may be subjected to heat and pressure to extract a resinous sap (“rosin”) containing cannabinoids; methods for obtaining rosin are well-known in the art. Other relevant methods to the formation of hashish are described in U.S. 63/025,863, U.S. 63/073,549, and U.S. 62/948,576, all of which are incorporated herein by reference.

The one or more additional component may thus be a cannabinoid in the form of a Cannabis crude extract, winterized extract, distillate, isolate, shatter, wax, or rosin.

In some embodiments, the one or more additional component may also be a terpene. As used herein, the term “terpene” generally refers to a class of chemical components comprised of the fundamental building block of isoprene, which can be linked to form linear structures or rings. Terpenes may include hemiterpenes (single isoprenoid unit), monoterpenes (two units), sesquiterpenes (three units), diterpenes (four units), sesterterpenes (five units), triterpenes (six units), and so on. At least some terpenes are expected to interact with, and potentiate the activity of, cannabinoids. Any suitable terpene may be used in the hybrid hashish product of the present disclosure. For example, terpenes originating from Cannabis plant may be used, including but not limited to aromadendrene, bergamottin, bergamotol, bisabolene, borneol, 4-3-carene, caryophyllene, cineole/eucalyptol, p-cymene, dihydrojasmone, elemene, farnesene, fenchol, geranylacetate, guaiol, humulene, isopulegol, limonene, linalool, menthone, menthol, menthofuran, myrcene, nerylacetate, neomenthylacetate, ocimene, perillylalcohol, phellandrene, pinene, pulegone, sabinene, terpinene, terpineol, 4-terpineol, terpinolene, and derivatives thereof. Additional examples of terpenes include nerolidol, phytol, geraniol, alpha-bisabolol, thymol, genipin, astragaloside, asiaticoside, camphene, beta-amyrin, thujone, citronellol, 1,8-cineole, cycloartenol, hashishene, and derivatives thereof. For example, the terpene may be hashishene, which is a class of terpenes found in hashish after mechanical processing. Without being bound by any theory, it is believed that hashishene may be responsible for the typical desirable “hashish flavour” that results from the degradation of a single terpene. Further examples of terpenes are discussed in US Patent Application Pub. No. US2016/0250270, which is herein incorporated by reference in its entirety for all purposes.

The hybrid hashish product of the present disclosure may contain one or more terpene(s). The one or more terpene(s) may originate from the isolated Cannabis trichomes, from the one or more additional component, or both. In some embodiments, the hybrid hashish product of the present disclosure may include the one or more terpene(s) in an amount (the “terpene content”) sufficient for the user to experience a desired entourage effect when consuming the product. For example, the hybrid hashish product may comprise a terpene content of from about 0.5 wt. % to about 15 wt. % terpene, the percentage being expressed relative to a total weight of the cohesive mass. For example, the hybrid hashish product may comprise a terpene content of up to about 15 wt. %, or up to about 10 wt. %, or up to about 5 wt. %, or up to about 4 wt. %, or up to about 3 wt. %, or up to about 2 wt. %, or up to about 1 wt. %.

In some embodiments, the one or more additional component may also be a flavonoid. The term “flavonoid” as used herein refers to a group of phytonutrients comprising a polyphenolic structure. Flavonoids are found in diverse types of plants and are responsible for a wide range of functions, including imparting pigment to petals, leaves, and fruit. Any suitable flavonoid may be used in the hybrid hashish product of the present invention. For example, flavonoids originating from a Cannabis plant may be used, including but not limited to: apigenin, cannflavin A, cannflavin B, cannflavin C, chrysoeril, cosmosiin, flavocannabiside, homoorientin, kaempferol, luteolin, myricetin, orientin, quercetin, vitexin, and isovitexin.

In some embodiments, the one or more additional component may be a flavoring agent. Any suitable flavoring agent known in the art may be used. For example, and without wishing to be limiting, the flavoring agent may be selected from the group consisting of extracts of cinnamon, monk fruit, cucumber, mint, orange, lime, citrus, cookie dough, chocolate, vanilla, jasmine, lychee, almond, banana, grape, pear, pineapple, pine, oak, apple, pumpkin, grapefruit, watermelon, cotton sugar, durian, longan, taro, sapote, toffee nut, caramel, lotus, mango, mangosteen, coconut, coffee, strawberry, passion fruit, blueberry, raspberry, kiwi, walnut, cocoa, cherimoya, custard apple, papaya, fig, plum, nectarine, peaches, guava, honeydew, jackfruit, kumquat, loquat, palm, pomelo, persimmon, quince, and tamarind, or any combinations thereof. Other examples of suitable flavoring agents include, but are not limited to, mint oils, wintergreen, clove bud oil, cassia, sage, parsley oil, marjoram, lemon, orange, propenyl guaethol, heliotropine, 4-cis-heptenal, diacetyl, methyl-p-tert-butyl phenyl acetate, methyl salicylate, ethyl salicylate, 1-menthyl acetate, oxanone, a-irisone, methyl cinnamate, ethyl cinnamate, butyl cinnamate, ethyl butyrate, ethyl acetate, methyl anthranilate, iso-amyl acetate, iso-amyl butyrate, allyl caproate, eugenol, eucalyptol, thymol, cinnamic alcohol, octanol, octanal, decanol, decanal, phenylethyl alcohol, benzyl alcohol, a-terpineol, linalool, limonene, citral, neral, geranial, geraniol nerol, maltol, ethyl maltol, anethole, dihydroanethole, carvone, menthone, beta-damascenone, ionone, gamma-decalactone, gamma-nonalactone, y-undecalactone, and any combinations thereof.

In some embodiments, the one or more additional component may be a coloring agent (also called “colorant”). Any suitable coloring agent known in the art may be used. For example, and without wishing to be limiting, the coloring agent may be any suitable food grade and/or non-toxic colorant or coloring agent known in the art.

The reader will readily understand that in embodiments of the present disclosure, the one or more additional component may include a combination of any one of the above examples of additional components.

### Shape of Hybrid Hashish Product

The hybrid hashish product as described herein may be formed or shaped. For example, the hybrid hashish products of the present disclosure may have an elongated body shape.

In some embodiments, the elongated body shape can be produced by forcing the mixture of the cohesive mass of isolated Cannabis trichomes and Cannabis plant biomass particles through the die of an extruder or plate of a food grinder. Due to the manufacturing process, the produced hybrid hashish products advantageously may have near-homogenous contents due to the mixing within the elongated enclosure, such that the isolated Cannabis trichomes are homogenized and the particles of the Cannabis plant biomass are uniformly distributed within the cohesive mass.

In some embodiments, the hybrid products can also have a substantially uniform cross-section along their lengths and near-identical appearance to others of the products produced within the same batch due to the uniformity of the shapes produced by the die.

In some embodiments, the hybrid hashish product may be an elongated three-dimensional shape, having an elongated body and a cross-section. The cross-section may be any desired shape; for example, the cross-section may have a simple solid shape such as, but not limited to a circle, an ellipse, a triangle, rectangle, square, pentagon, hexagon, or any other polygonal shape, or a more complex shape. The shape of the cross-section can be solid or hollow; for example, the cross-section may be an annulus and the resulting shape of the hybrid hashish product is a tube; in another example, the cross-section may be a circle and the resulting shape of the hybrid hashish product is a cylinder.

In some embodiments, the elongated body of the hybrid hashish product has a substantially uniform cross-section along at least a portion of its length. By the term “substantially uniform”, it is meant that the width of the cross-section remains relatively constant along at least a portion of the length of the elongated body.

In some embodiments, the elongated body of the hybrid hashish product comprises a cross-section having a width that is within a 5% margin, preferably within a 2% margin of an average cross-section width along a portion of the length of the elongated body or along the entire length of the elongated body. In other words, the elongated body comprises a cross-section width at one location along the at least portion of the length which can be characterized as representing a ratio of from about 0.90 to about 1.10, or from about 0.95 to about 1.05, or from about 0.98 to about 1.02 when compared to an average cross section width of the full length of the body or of the at least portion of the length of the body, where the ratio is cross section width/average cross section.

As would be understood by a person of skill in the art, the width (also referred to as the “diameter”) of the cross-section of the hashish product may depend on the application for its use. In some embodiments, the elongated body of the hybrid hashish product may have a cross-section along at least a portion of its length having a width of from about 1 mm to about 25 mm, including any ranges there in-between or any values therein. For example, a width of from about 1 mm to about 10 mm. For example, a width of about 1 mm, or about 1.5 mm, or about 2.0 mm, or about 2.5 mm, or about 3.0 mm, or about 3.5 mm, or about 4.0 mm, or about 4.5 mm, or about 5.0 mm, or about 5.5 mm, or about 6.0 mm, or about 6.5 mm, or about 7.0 mm, or about 7.5 mm, or about 8.0 mm, or about 8.5 mm, or about 9.0 mm, or about 9.5 mm, or about 10.0 mm, or about 10.5 mm, or about 11.0 mm, or about 11.5 mm, or about 12.0 mm, or about 12.5 mm, or about 13.0 mm, or about 13.5 mm, or about 14.0 mm, or about 14.5 mm, or about 15.0 mm, or about 16.0 mm, or about 17.0 mm, or about 18.0 mm, or about 19.0 mm, or about 20.0 mm, or about 21.0 mm, or about 22.0 mm, or about 23.0 mm, or about 24.0 mm, or about 25.0 mm, or even more in some embodiments. With respect to the ratio of the cross-section width at one location along at least portion of the length of the elongated body of the hashish product discussed above, the reader will thus understand that when the average cross-section width has a value of x, e.g., 10 mm, then the cross-section width at one location along at least portion of the length may have a value of x+/−10%, e.g., from 9 mm to 11 mm.

The weight of the hybrid hashish product of the present disclosure will be dependent on the size and shape of the cross-section and the length of the elongated body (as discussed below). In some embodiments, the length of the elongated body may be determined by the intended use of the product.

For example, when the hashish product is intended to form a cigarette-like or cigar-like smoking article, the weight thereof may be of from about 1 g to about 20 g, including any ranges there in-between or any values therein, or even more. For example, the hashish product may have a total weight of about 1.0 g, about 1.25 g, about 1.5 g, about 1.75 g, about 2.0 g, about 2.25 g, about 2.75 g, about 3.0 g, about 3.5 g, about 4.0 g, about 4.5 g, about 5.0 g, about 5.5 g, about 6.0 g, about 6.5 g, about 7.0 g, about 7.5 g, about 8.0 g, about 8.5 g, about 9.0 g, about 9.5 g, about 10.0 g, about 10.5 g, about 11.0 g, about 11.5 g, about 12.0 g, about 12.5 g, about 13.0 g, about 13.5 g, about 14.0 g, about 14.5 g, about 15.0 g. The reader will appreciate that a hashish product intended to form a cigarette-like or cigar-like smoking article may have a weight which is suitable for that purpose other than those values listed here.

For example, when the hashish product include notches or contains multiple servings (as described later in this text), the hashish product may have a total weight of from about 0.2 g to about 20 g, including any ranges there in-between or any values therein. For example, the hashish product may have a total weight of 0.2 g, about 0.5 g, about 1.0 g, about 1.5 g, about 2.0 g, about 2.5 g, about 3.0 g, about 3.5 g, about 4.0 g, about 4.5 g, about 5.0 g, about 5.5 g, about 6.0 g, about 6.5 g, about 7.0 g, about 7.5 g, about 8.0 g, about 8.5 g, about 9.0 g, about 9.5 g, about 10.0 g, about 10.5 g, about 11.0 g, about 11.5 g, about 12.0 g, about 12.5 g, about 13.0 g, about 13.5 g, about 14.0 g, about 14.5 g, about 15.0 g, about 15.5 g, about 16.0 g, about 16.5 g, about 17.0 g, about 17.5 g, about 18.0 g, about 18.5 g, about 19.0 g, about 19.5 g, or about 20.0 g. Each serving in the multiple servings may be portioned from the hashish product such that each serving may have a suitable weight, for example of from about 0.2 g to about 5.0 g, or even more. The reader will appreciate that a hashish product intended to include notches or contains multiple servings may have a total weight or a weight for each single unit that is suitable for that purpose other than those values listed here.

In some cases, a variability of about +/−10% on the measured weight between hybrid hashish product units in a batch of hybrid hashish products, or between the measured weight of a first portion of the hybrid hashish product and an average weight of the hybrid hashish product can be acceptable to the manufacturer.

The hybrid hashish product of the present disclosure may be loaded into a rolling medium or wrapper to form a smoking article. It will be apparent that such loading may be performed at the manufacturing site or by an end-user. The rolling medium or wrapper may be any suitable rolling medium or wrapper known in the art, for example cigarette-type rolling paper or cigar-like wrapper. The rolling medium or wrapper can be made with a material such as paper, hemp, cordia palm leaf, tendu leaf, flower petal, banana leaves, flax, sisal, rice straw, esparto, Cannabis leaves, and the like, and may be transparent, colored and/or flavored. When desired, the rolling medium or wrapper may also further include an additive on one of its surfaces (internal or external), such as kief, terpenes, Cannabis distillate, and the like. The hybrid hashish product of the present disclosure may, if desired, be loaded into the rolling medium or wrapper along with other smokable Cannabis products, such as Cannabis flower, kief, Cannabis distillate, Cannabis rosin (a solid form of resin produced by heating fresh liquid resin to vaporize the volatile liquid terpene component), Cannabis resin, Cannabis wax, Cannabis shatter (a translucent butane hash oil extract that looks like amber and has a consistency almost like hard candy butter) or other smokable materials, such as tobacco leaves.

As would be understood by a person of skill in the art, the length of the hashish product may depend on the application for its use and/or the form of the compounded hashish product produced.

The hybrid hashish product described herein may have a length that is configured to be approximately that one of a standard Cannabis cigarette (also called a “joint”) length, or for multiple servings of such product. For example, the length of the hybrid hashish product may be from about 40 mm to about 300 mm, such as 40 mm to about 300 mm, or from about 50 mm to about 140 mm, or from about 60 mm to about 130 mm, or from about 70 mm to about 120 mm, or from about 80 mm to about 110 mm, or any values in between these values. More specifically the hybrid hashish product may be about 40 mm, 45 mm, 50 mm, 55 mm, 60 mm, 65 mm, 70 mm, 75 mm, 80 mm, 85 mm, 90 mm, 95 mm, 100 mm, 105 mm, 110 mm, 115 mm, 120 mm, 125 mm, 130 mm, 135 mm, 140 mm, 145 mm, 150 mm, 155 mm, 160 mm, 165 mm, 170 mm, 175 mm, 180 mm, 185 mm, 190 mm, 195 mm, 200 mm, 205 mm, 210 mm, 215 mm, 220 mm, 225 mm, 230 mm, 235 mm, 240 mm, 245 mm, 250 mm, 255 mm, 260 mm, 265 mm, 270 mm, 275 mm, 280 mm, 285 mm, 290 mm, 295 mm, or about 300 mm, in length. In some embodiments the length may be approximately about 50 mm, or about 75 mm.

The hybrid hashish product of the present disclosure may have a shape such that upon being loaded into the rolling medium or wrapper (as described above), one forms a smoking article with a hollow core. The hollow core can have a diameter that is approximately 50% of the diameter of the entire hashish product, e.g., between about 30% and 70%. In this embodiment, the hybrid hashish product may have a length that is configured to be approximately that one of a standard Cannabis/hashish cigarette length, or for multiple servings of such product. For example, the length of the hybrid hashish product may be from about 40 mm to about 300 mm, such as about 40 mm to about 300 mm, or from about 50 mm to about 140 mm, or from about 60 mm to about 130 mm, or from about 70 mm to about 120 mm, or from about 80 mm to about 110 mm, or any values in between these values. More specifically the hybrid hashish product may be about 40 mm, about 45 mm, about 50 mm, about 55 mm, about 60 mm, about 65 mm, about 70 mm, about 75 mm, about 80 mm, about 85 mm, about 90 mm, about 95 mm, about 100 mm, about 105 mm, about 110 mm, about 115 mm, about 120 mm, about 125 mm, about 130 mm, about 135 mm, about 140 mm, about 145 mm, about 150 mm, about 155 mm, about 160 mm, about 165 mm, about 170 mm, about 175 mm, about 180 mm, about 185 mm, about 190 mm, about 195 mm, about 200 mm, about 205 mm, about 210 mm, about 215 mm, about 220 mm, about 225 mm, about 230 mm, about 235 mm, about 240 mm, about 245 mm, about 250 mm, about 255 mm, about 260 mm, about 265 mm, about 270 mm, about 275 mm, about 280 mm, about 285 mm, about 290 mm, about 295 mm, or about 300 mm, in length. In some embodiments the length may be approximately 50 mm, or about 75 mm. In this embodiment, the hybrid hashish product may have a hollow core that is the full length of the hybrid hashish product. Alternatively, the hybrid hashish product may have a hollow core that is less than the full length of the hybrid hashish product, e.g., is 90% of the full length, or 80% of the full length, or even less.

In another embodiment, the hybrid hashish product of the present disclosure may be mounted to a smoking device, for example to be used in a heat-not burn device. Such devices are known in the art and one aim of such heated smoking articles is to reduce known harmful smoke constituents of the type produced by the combustion and pyrolytic degradation of smoking material in conventional Cannabis cigarettes. Typically, in heat-not-burn device, an aerosol is generated by the transfer of heat from a heat source to a physically separate aerosol-forming substrate or material, which may be located within, around or downstream of the heat source. During smoking, volatile compounds are released from the aerosol-forming substrate by heat transfer from the heat source and entrained in air drawn through the smoking article. As the released compounds cool, they condense to form an aerosol that is inhaled by the user. Examples of heat-not-burn devices which may be suitable for mounting the hashish product of the present disclosure include but are not limited to the Omura™ (Omura), iQOS™ (Philip Morris International), and Glo™ (British American Tobacco), and PAX™ (PAX Labs). In such an embodiment, the hashish product may be mounted to a suitable mounting component for use in such smoking device, such as a mounting cartridge or tube, alone or along with other smokable Cannabis products and/or smokable materials, such as those described previously. The hybrid hashish product described herein may have a length that is configured to be approximately that one of a standard stick or cartridge for such devices. For example, the length of the hybrid hashish product may be from about 50 mm to about 120 mm; more specifically the hashish product may be about 70 mm, about 75 mm, about 80 mm, about 85 mm, about 90 mm, about 95 mm, about 100 mm, about 105 mm, about 110 mm, about 115 mm, or about 120 mm in length. Alternatively, the hybrid hashish product may be approximately 50 mm, or about 75 mm.

As discussed above, the weight of the hybrid hashish product of the present disclosure will be dependent on the size and shape of the cross section and the length of the elongated body. In embodiments where the hybrid hashish product is configured to be the length of a Cannabis cigarette or for use in a heat-not-burn device, the hybrid hashish product may have a weight of from about 0.2 g to about 3.5 g. For example, the hashish product may have a weight of about 0.2 g, about 0.5 g, about 0.6 g, about 0.75 g, about 1.0 g, about 1.25 g, about 1.5 g, about 1.75 g, about 2.0 g, about 2.25 g, about 2.75 g, about 3.0 g, about 3.25 g, or about 3.5 g.

Use of Hashish Products

Hashish products are typically used for recreational and/or medicinal uses.

For example, hashish products can be used to achieve a desired effect in a user, such as a psychoactive effect, a physiological effect, or a treatment of a condition. By “psychoactive effect”, it is meant a substantial effect on mood, perception, consciousness, cognition, or behavior of a subject resulting from changes in the normal functioning of the nervous system. By “physiological effect”, it is meant an effect associated with a feeling of physical and/or emotional satisfaction. By “treatment of a condition”, it is meant the treatment or alleviation of a disease or condition by absorption of cannabinoid(s) at sufficient amounts to mediate the therapeutic effects.

The terms “treating”, “treatment” and the like are used herein to mean obtaining a desired pharmacologic and/or physiologic effect. The effect may be prophylactic, in terms of completely or partially preventing a disease, condition, or symptoms thereof, and/or may be therapeutic in terms of a partial or complete cure for a disease or condition and/or adverse effect, such as a symptom, attributable to the disease or disorder. “Treatment” as used herein covers any treatment of a disease or condition of a mammal, such as a dog, cat or human, preferably a human.

In certain embodiments, the disease or condition is selected from the group consisting of pain, anxiety, an inflammatory disorder, a neurological disorder, a psychiatric disorder, a malignancy, an immune disorder, a metabolic disorder, a nutritional deficiency, an infectious disease, a gastrointestinal disorder, and a cardiovascular disorder. Preferably the disease or condition is pain. In other embodiments, the disease or condition is associated with the feeling of physical and/or emotional satisfaction.

In the context of recreational use, the “effective amount” administered and rate and time-course of administration, will depend on the desired effect associated with a feeling of physical and/or emotional satisfaction in the subject.

In the context of health and wellness use, the “effective amount” administered, and rate and time-course of administration will depend on the nature and severity of the disease or condition being treated and typically also takes into consideration the condition of the individual subject, the method of administration and the like.

### Manufacturing Process—Mixing Overview

The hybrid hashish product of the present disclosure may be produced by mixing raw materials for making the hybrid hashish product (e.g., isolated trichomes and Cannabis plant biomass) under conditions sufficient to obtain a cohesive mass.

For example, the mixing may be performed by mechanically mixing. By the term “mechanically mixing” or “mechanical mixing”, it is meant mixing using any suitable mechanical means. The mechanical means may be a plurality of interpenetrate helicoidal surfaces within an elongated enclosure, a non-limiting example of which is a food grinder, or an industrial extruder apparatus.

A food grinder (also called meat grinder) is typically a home kitchen appliance or a commercial apparatus for fine chopping (“mincing”) and/or mixing of raw or cooked meat, fish, vegetables, or similar foods. The food to be minced is placed into a funnel, which sits on top of the grinder. From there, the material enters a horizontal screw conveyor; the screw conveyor may be hand-cranked or powered by an electric motor. The screw squashes and mixes the food. At the end of the screw, the food is passed through a fixed plate, where it exits the machine. The fineness of the minced food depends on the size of the holes in the plate. For example, a food grinder can be the KSMMGA metal food grinder attachment (KitchenAid™, Canada) or the G1700 (Gvode, Inc.).

An industrial extruder apparatus is used to perform the extrusion process. Industrial extruders typically have a tubular barrel, usually electrically heated; a revolving screw, ram, or plunger within the barrel; a hopper at one end from which the material to be extruded is fed to the screw, ram or plunger; a die at the opposite end for shaping the extruded mass. Using a system of barrels or cylinders containing interpenetrate helicoidal surfaces, e.g., screw pumps or extruder screws, the extruder apparatus mixes the product while heating it and propels it through the die to create the desired shape.

An industrial extruder apparatus can be a single screw extruder or a twin-screw extruder. For example, a single screw extruder can be the ETP1 lab extruder (The Bonnot Company, United States) and a twin-screw extruder can be the Process 11 Parallel Twin-screw Extruder (Thermo Fisher Scientific, United States). An industrial extruder apparatus can be configured to have one or more mixing zones, one or more temperature zones, and one or more input zones. The input zones are used for introduction of material, for example introduction of the isolated Cannabis trichomes and particles of the Cannabis plant biomass as well as introduction of additional components (e.g., cannabinoids, terpenes, flavonoids, water, flavoring agents, coloring agents, etc.). The mixing zones apply compression and shear forces to the input materials, blending until they are homogenized.

The extruder may include a die assembly that may perform a variety of functions: it may form or shape the extrudate, it may divide the extrudate into multiple extrudates, it may inject an additive substance into the extrudate, and it may compress and reduce the cross-sectional area of the extrudate. Single screw extruders are known in the art—the screws of such extruders comprise grooves and may be cylindrical, conical, tapered and the likes as described for example in CA 2,731,515, U.S. Pat. No. 6,705,752, CN101954732 and CN201792480, where each of which is herein incorporated by reference in its entirety. Twin screw extruders are also known in the art—screws of such extruders may be parallel or non-parallel, converging or non-converging, with or without differential speed, counter or non-counter rotating as described for example in U.S. Pat. No. 6,609,819, WO 2020/220390, WO 2020/220495 and US 2010/0143523, where each of which is herein incorporated by reference in its entirety. Single screw and twin-screw arrangements may also be integrated within a single extruder device, as described for example in U.S. Pat. No. 10,124,526, which is herein incorporated by reference in its entirety. It will be readily appreciated that extruders have flexible configuration (in terms of mixing zones, temperature zones, input zones, etc.) and that any suitable configuration of the extruder apparatus that produces the hash product may be used within the context of the present disclosure.

Feeding the materials into the food grinder or extruder, may include a starve feeding step, for example.

The mixing can be applied to the raw materials for making the hybrid hashish product (e.g., the isolated Cannabis trichomes and the Cannabis plant biomass) within the elongated enclosure under conditions sufficient to obtain a cohesive mass.

The conditions or variables that can be modified during production include one or more of temperature, screw rotation speed, and pressure.

### Manufacturing Hybrid Hashish Products

Various methods for making a hybrid hashish product are described. For example, the method includes providing raw materials (e.g., isolated Cannabis trichomes and Cannabis plant material biomass), and mixing the raw materials under conditions sufficient to obtain a cohesive mass comprising agglomerated Cannabis trichomes and particles of the Cannabis plant material biomass. When the mixing apparatus used for mixing includes an apparatus such as a food grinder, the raw materials advantageously include isolated Cannabis trichomes and Cannabis plant biomass that have been pre-treated (i.e., the raw materials are “pre-treated raw materials”, as will be further described later in this text). Alternatively, when the mixing apparatus used for mixing includes an apparatus capable of imparting higher shearing and/or thermal energy, such as an industrial extruder, it is not necessary to use raw materials that have been pre-treated.

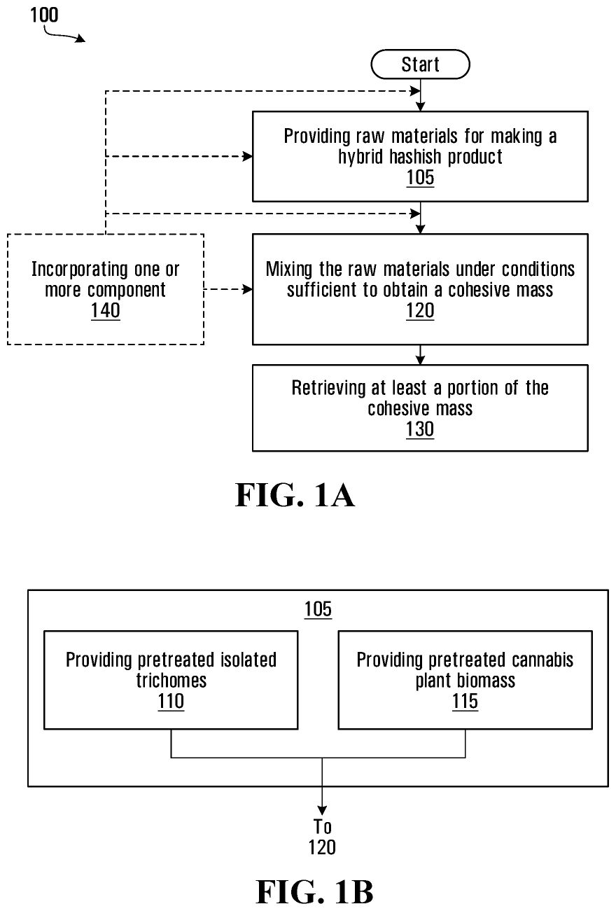

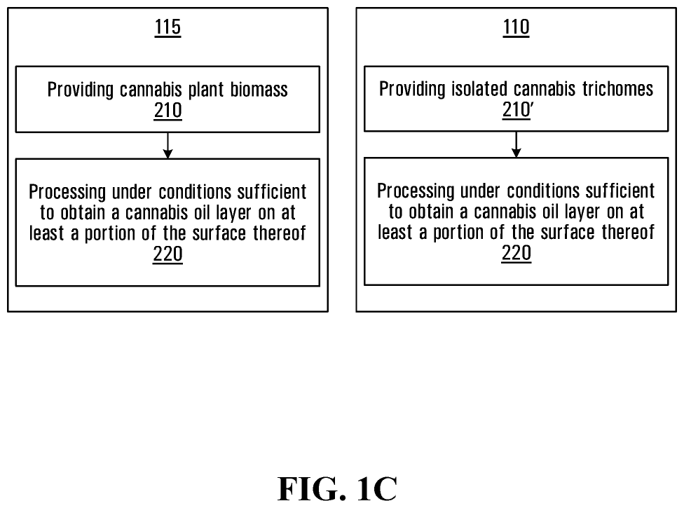

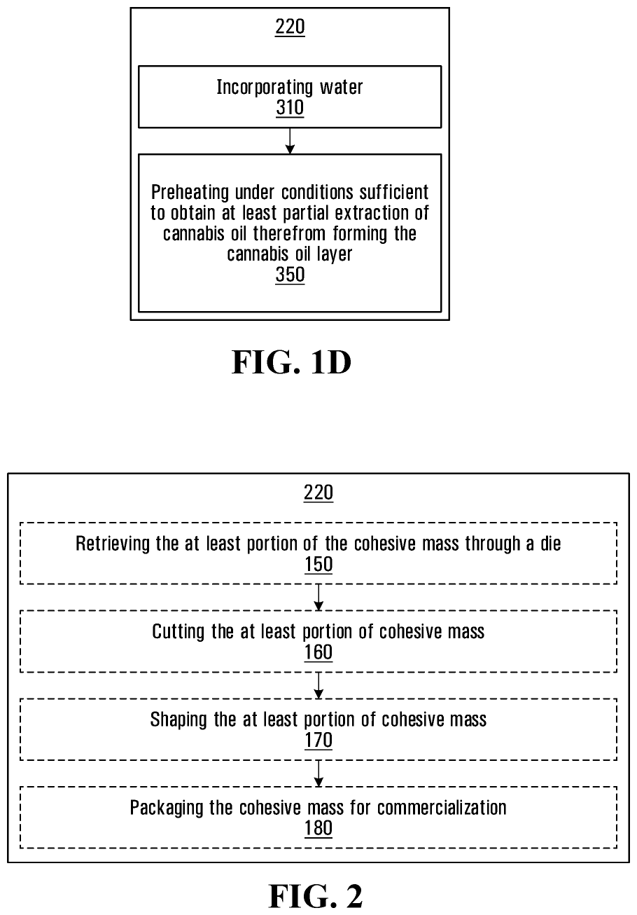

FIG. 1 shows a non-limiting example of a process 100 for producing a hybrid hashish product, when using an apparatus such as a food grinder. The process 100 includes providing raw materials at step 105.

In one embodiment, the step 105 comprises providing isolated pre-treated Cannabis trichomes at step 110 and providing pre-treated Cannabis plant biomass at step 115, where both steps 110 and 115 may be separate steps performed sequentially or may be combined as a single step, for example. As discussed above, when using an apparatus capable of imparting higher shearing and/or thermal energy, such as an industrial extruder, the step 105 comprises instead providing isolated Cannabis trichomes at step 110 and providing Cannabis plant biomass at step 115, where both steps 110 and 115 may be separate steps performed sequentially or may be combined as a single step, for example.

For sake of conciseness, the following text will focus on isolated pre-treated Cannabis trichomes and pre-treated Cannabis plant biomass. The reader will, however, understand that that the pretreatment steps are not necessary in case the mixing is performed with an apparatus capable of imparting higher shearing and/or thermal energy, such as an industrial extruder.

The producer implementing the process 100 may obtain the pre-treated isolated Cannabis trichomes of step 110 and/or pre-treated Cannabis plant biomass of step 115 from another producer. The step 105 may thus include a sub-step of obtaining the pre-treated isolated Cannabis trichomes and/or pre-treated Cannabis plant biomass from another producer (not shown in figures).

Alternatively, the producer implementing the process 100 may obtain the pre-treated isolated Cannabis trichomes of step 110 and/or pre-treated Cannabis plant biomass of step 115 via at least the following steps.

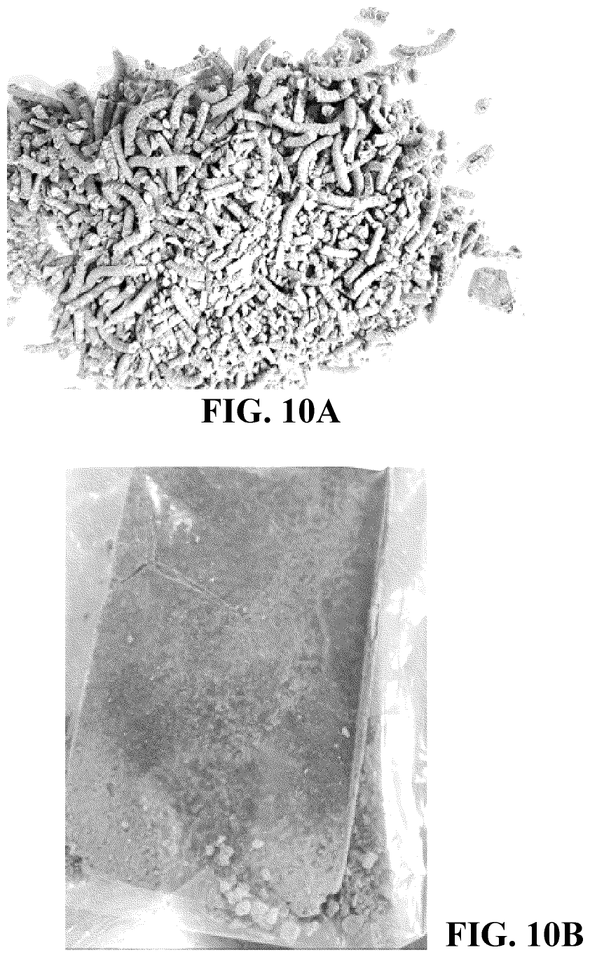

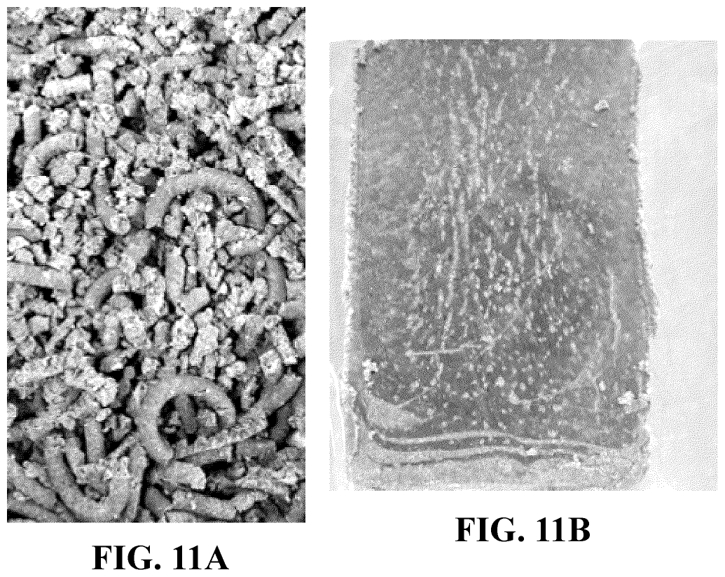

FIG. 10 shows an example of obtaining pre-treated raw materials.

With respect to step 115, there is a first step 210 of providing Cannabis plant biomass. The Cannabis plant biomass may comprise Cannabis flowers/buds, Cannabis trim, Cannabis leaves, or any combination thereof. The producer implementing this step 210 may also produce the Cannabis plant biomass or may obtain the Cannabis plant biomass from another producer. In a second step 220, the Cannabis plant biomass is processed under conditions sufficient to obtain a Cannabis oil layer on at least a portion of the surface thereof.

With respect to step 110, there is a first step 210′ of providing isolated Cannabis trichomes. The producer implementing this step 210′ may also produce the isolated Cannabis trichomes or may obtain the isolated Cannabis trichomes from another producer. In a second step, the isolated Cannabis trichomes are pre-treated with the processing step 220 described above.

In some embodiments, the steps 210 and/or 210′ may be performed at a first location while the remaining steps of process 100 may be performed at a second location, where the first and second locations may be within the same licensed producer site or within different licensed producer sites. In some embodiments, all steps of process 100 may be performed at the same location.

The processing step 220 can be performed at least as illustrated in FIG. 1D.

Processing step 220 includes incorporating water into the materials being pre-treated at step 310. The person of skill will readily appreciate that water could be incorporated in the form of steam, liquid, ice, or any combination thereof. The materials being pre-treated can be isolated Cannabis trichomes and/or Cannabis plant biomass, depending on specific implementations. For example, the materials being pre-treated can include a mixture of isolated Cannabis trichomes and Cannabis plant biomass to which water is incorporated at step 310. The water incorporated may be distilled, reverse osmosis and/or microfiltered water. In some embodiments, water may be incorporated to have a total water content of about 20 wt. % or less. For example, a total water content of from about 5 wt. % to about 15 wt. % or any value therebetween, or in a range of values defined by any values therebetween. For example, a total water content of about 15 wt. % or less, about 14 wt. % or less, about 13 wt. % or less, about 12 wt. % or less, about 11 wt. % or less, about 10 wt. % or less. For example, a total water content of from about 10 wt. % to about 15 wt. %, from about 10 wt. % to about 12 wt. %.

Processing step 220 further includes a step 350 of preheating for sufficient time to ooze Cannabis oil out from the Cannabis trichomes and/or Cannabis plant biomass and obtain a Cannabis oil layer on the at least portion of the surface thereof.

For example, the preheating can be performed at a temperature of from about 70° C. to about 130° C., preferably from about 80° C. to about 125° C., more preferably from about 120° C. to about 125° C.

For example, the preheating is performed for a duration of from about 10 minutes to about 60 minutes, preferably from about 30 minutes to about 40 minutes.

In some embodiments, the preheating is performed in a sealed container. Performing the preheating in a sealed container can be advantageous to reduce/minimize terpene loss during the preheating treatment. This is because in a sealed container, with increasing temperatures, the Cannabis material will release CO₂ from decarboxylation and the water incorporated into the material will vaporize to steam, thus increasing pressure. With an increased pressure inside the sealed container, the boiling point of terpenes will increase such that less terpenes will evaporate from the Cannabis material resulting in a pretreated Cannabis material that retains most of the terpene profile present prior to the pre-treatment. Further, the presence of water and steam within the sealed container will also result in more efficient heat transfer to the Cannabis material, thus resulting in more efficient oozing out of Cannabis oil. As an additional advantage, the cannabinoids contained in the Cannabis material will also benefit from such increased heat transfer efficiency in having better decarboxylation levels.

The isolated trichomes may have been isolated from a single Cannabis plant strain or from a plurality of Cannabis plant strains that may have different respective cannabinoid concentrations. All are viable options. It is known amongst consumers of hashish and other Cannabis products that using isolated Cannabis trichomes produced from more than one strain of Cannabis plant allows a user to tune the psychoactive and/or entourage effect obtained by consuming the product. The mixing of Cannabis plant strains may also allow adjustments to the final concentration of a component of the product, for example but not limited to the cannabinoid content. Additionally, use of more than one Cannabis strain allows for improved product and waste management—important in commercial production. The isolated Cannabis trichomes added in step 110 of process 100 can be dry-sift kief.

The Cannabis plant biomass may have been harvested from a single Cannabis plant strain or from a plurality of Cannabis plant strains that may have different respective cannabinoid concentrations. All are viable options. The Cannabis plant biomass can be from any part of the Cannabis plant, for example, they can be one or more of flowers, buds, sugar leaves, or trim.

The pre-treated Cannabis plant biomass provided at step 115 can be in the form of particles that can have been made by mechanical processes, such as with an ordinary coarse pulveriser capable of grinding or milling Cannabis plant biomass into particles. There are no particular limitations on the pulverization step, and the average particle size of the pulverized Cannabis plant biomass can be in the range of several hundreds of micrometers to several millimeters.

Alternatively, the pre-treated Cannabis plant biomass provided at step 115 is in a whole form (i.e., whole flowers, sugar leaves, buds, or trim) or partially shredded. Optionally, the pre-treated Cannabis plant biomass has been dried (cured). During mixing at step 120, the mixing action can break up the Cannabis plant biomass into smaller pieces, and optionally, the “movement” of the trichomes during mixing can also contribute to the breakup of the Cannabis plant biomass.

The particles of the Cannabis plant biomass can be in the range of several hundreds of micrometers to several millimeters. For example, the particles of the Cannabis plant biomass can have a size that passes through a sieve having a 10.0 mm pore size or less. For example, the particles can have a size of between about 0.1 mm and about 10 mm, including any values therein. Preferably, the particles of the Cannabis plant biomass have a size that passes through a sieve having a 2.0 mm pore size.

The Cannabis plant biomass provided at step 115 represents from about 5 wt. % to about 50 wt. %, where the percentage is expressed relative to the total weight of the raw materials for making the hybrid hashish product. For example, the pre-treated Cannabis plant biomass provided at step 115 may represent an amount of about 10 wt. % to about 50 wt. %, where the percentage is expressed relative to the total weight of the raw materials for making the hybrid hashish product, or any value therebetween, or in a range of values defined by the aforementioned values. For example, the pre-treated Cannabis plant biomass provided at step 115 may represent an amount of about 10 vol. %, about 15 vol. %, about 20 vol. %, about 25 vol. %, about 30 vol. %, about 35 vol. %, about 40 vol. %, about 45 vol. %, or about 50 vol. %, where the percentage is expressed relative to the total weight of the raw materials for making the hybrid hashish product.

### Mixing Raw Materials

At step 120, the raw materials are mixed under conditions sufficient to obtain a cohesive mass comprising isolated trichomes and particles of Cannabis plant biomass.

The conditions at the mixing step 120 comprise one or more of shear, pressure, and temperature, which may be varied to alter the characteristics of the hybrid hashish product. The characteristics that are altered may include, but without being limited to, stickiness, dryness, color, tactual characteristics, ductility (i.e., characteristic that defines the level of malleability of the hashish product), hardness or resistance to localized deformation (i.e., characteristic that determines how easy it is to cut or separate the hashish product), toughness (i.e., characteristic that determines the likelihood that the hashish product deforms rather than fractures under an applied force), and the like.

For example, the temperature being applied at the mixing step 120 may be at a temperature of about 170° C. or less. For example, a temperature of from about 20° C. to about 170° C., including any ranges therein or any value therein. For example, a temperature of about 20° C., about 25° C., about 30° C., about 35° C., about 40° C., about 45° C., about 50° C., about 55° C., about 60° C., about 65° C., about 70° C., about 80° C., about 90° C., about 100° C., about 110° C., about 120° C., about 130° C., about 140° C., about 150° C., about 160° C., or about 170° C. It will be readily apparent to the person skilled in the art that different temperatures corresponding to the abovementioned temperature values or ranges may be used in different zones during the process as described elsewhere in this text.

For example, the pressure being applied at the mixing step 120 may be at a value of about 1 bar or more. For example, a pressure of from about 1 bar to about 70 bar, including any ranges therein or any value therein. For example, a pressure of from about 1 bar to about 65 bar, from about 3 bar to about 60 bar, from about 4 bar to about 55 bar, from about 6 bar to about 50 bar, from about 8 bar to about 45 bar, from about 10 bar to about 40 bar, from about 12 bar to about bar, from about 12 bar to about 32 bar, from about 14 bar to about 30 bar, from about 16 bar to about 28 bar, from about 18 bar to about 26 bar, or from about 20 bar to about 24 bar including any ranges therein or any value therein. For example, a pressure of about 1 bar, about 3 bar, about 4 bar, about 5 bar, about 8 bar, about 10 bar, about 12 bar, about 14 bar, about 16 bar, about 18 bar, about 22 bar, about 22 bar, about 24 bar, about 26 bar, about 28 bar, about 30 bar, about 32 bar, about 35 bar, about 40 bar, about 45 bar, about 50 bar, about 55 bar, about 60 bar, or around 65 bar. The person of skill will readily understand that a given pressure value may be selected depending on the die that is used to form the hashish product, as described elsewhere in this text.

In a practical implementation, the mixing includes applying compression and shear forces to the pre-treated raw materials (e.g., pre-treated isolated trichomes and pre-treated Cannabis plant biomass) via a plurality of interpenetrate helicoidal surfaces within an elongated enclosure. Preferably, the elongated enclosure is an extruder device having at least one screw or a food grinder. The mixing shear and compressive forces can be controlled by modulating the rotational speed of at least one screw within the extruder or the food grinder. In such embodiments, the screw rotation per minute (rpm) can be selected to perform the mixing step 120 at a value of for example about 10 rpm or more. For example, the screw rpm can be selected in a range of from about 15 to about 1000 rpm, such as from about 20 to about 500 rpm, or from about 25 to about 450 rpm, or from about 30 to about 400 rpm, or from about 45 to about 450 rpm including any value within any of these ranges.

The mixing step 120 can continue until a desired level of homogeneity is obtained. For example, step 120 can continue for a minimum time, e.g., about 30 minutes, about 20 minutes, or about 10 minutes depending on the elongated enclosure length and the speed of the screw. In some instances, the mixing time can be between about 15 seconds and about 5 minutes, e.g., from about 20 seconds to about 1 minute. In some embodiments, the mixing step 120 can be repeated, e.g., materials can be mixed in the food grinder, recovered and reloaded into the food grinder for more than one pass into the food grinder, until achieving the desired level of homogeneity and/or texture.

In some embodiments where the mixing step 120 is performed in an extruder having at least one screw, the residence time within the extruder barrel is directly related to the length of the barrel and the rotational speed of the at least one screw. To increase mixing time of the components within the barrel, the components can travel through at least one zone of the barrel in a distal direction, and then be redirected to at least one zone of the barrel in a proximal direction (i.e., towards the inlet rather than towards the die).

Optional step 140 includes incorporating one or more additional component(s) at one or more steps during the process 100. For example, one or more additional component(s) can be added prior to, simultaneously with, or following step 105, or prior to, simultaneously with, or following the mixing step 120. Multiple additional components may be added in a single step or may be added separately in one or more consecutive steps or at different times or points along the process 100. The one or more additional components can be a cannabinoid, a terpene, a flavonoid, a flavoring agent, a coloring agent, or any combinations thereof. When the one or more component comprises a cannabinoid, the cannabinoid may be provided in the form of a Cannabis extract (including a crude extract, or a winterized extract), a distillate, an isolate, Cannabis rosin, Cannabis resin, Cannabis wax, or Cannabis shatter.

For example, the one or more additional components added in step 140 can be beneficially uniformly distributed in each hybrid hashish product.

Once sufficiently mixed to form a cohesive mass (e.g., after multiples passes in a food grinder), at least a portion of the cohesive mass is retrieved to obtain the hybrid hashish product at step 130.

With reference to FIG. 2, step 130 may include one or more optional steps 150, 160, 170, and 180.

For example, at step 150, at least a portion of the cohesive mass can be retrieved as it passes through a die assembly. The die assembly can be configured to impart a pre-determined shape to the cohesive mass. The size and shape imparted to the cohesive mass may be any desired shape, which will be determined by the size and shape of the perforations in the die. For example, and without wishing to be limiting in any manner, the cohesive mass may be shaped into an elongated product with a cross-section that is a circle, a triangle, rectangle, square, pentagon, hexagon, any other polygonal shape, or any other more complex design. The cross-section can be solid or hollow. In another example, the cohesive mass may be formed to have a shape that elongate, curved, shell-like, or other shape like pasta.

For example, at step 160 the cohesive mass may be subjected to a transverse cutting operation to cut it as desired. The shape and size of the resulting hybrid hashish product will be dependent on the shape of the die and how the product is cut. The cohesive mass may be cut according to a pre-determined cutting pattern, a pre-determined weight, or a pre-determined length to obtain smaller units of hybrid hashish product for a pre-determined packaging size. The finished hybrid hashish product may be of a size that is suitable for multiple portions of hybrid hashish (that is, a user may remove a desired portion size for each use) or may be a size suitable for a single use (that is, a ready-to-use single-use product).

For example, at step 170, the cohesive mass can be further shaped and formed. The further shaping and forming can be for ease of use, or to make an interesting experience for the consumer, as discussed in more detail below.

Finally, following any shaping, cooling, or other manufacturing steps, the cohesive mass can be further processed to prepare a hybrid product ready for commercialization, for example the cohesive mass can be packaged in step 180.

### Hybrid Hashish Products

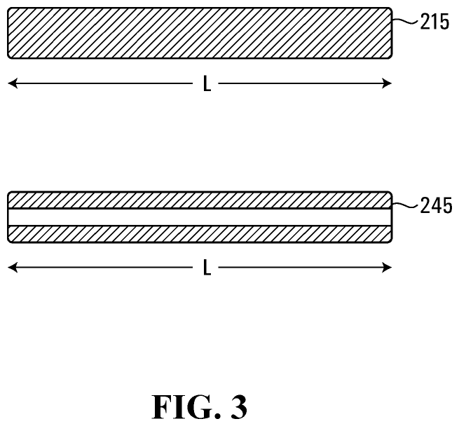

Referring to FIG. 3, various hybrid hashish products made with extruded body shapes can be produced with the methods of FIGS. 1 and 2. Each of the hybrid hashish products generally have an elongated body and a length, with the elongated body having a substantially uniform cross section along at least a portion of the length, with various cross sections possible. For example, and by way of illustration the cross section is a circle and the resulting shape of the elongated hybrid hashish product 215 is a cylinder, as shown in FIG. 3. Other possible cross sections include a triangle and a square. Any other simple solid cross-section is also possible, e.g., a rectangle, pentagon, hexagon, or any other polygonal shape. More complex designs are also possible, such as a star, or logo. Cross-sections that are not solid are also possible. For example, the cross-section can be an annulus and the resulting shape of the elongated hybrid hashish product 245 a tube, as also shown in FIG. 3.

Each of the elongated hybrid hashish products such as elongated hybrid hashish product 215 and 245 has a length L. In some embodiments, the length L is configured to be approximately that of a standard Cannabis cigarette length, for example from about 70 mm to about 120 mm. In such configurations, the elongated hybrid hashish products such as 215 and 245 are ready-to-use products. The user or the manufacturer can load the elongated hybrid hashish product 215, 245 along with one or more components into a rolling medium or wrapper (e.g., cigarette-like paper or cigar-like wrapper as discussed above) to form a hybrid hashish smoking article ready to be smoked by the user.

In some embodiments, the elongated hybrid hashish product 215, 245 is added along with additional components to the rolling medium or wrapper, e.g., kief, Cannabis trim, sugar leaves, or tobacco leaves.

Alternatively, the hybrid hashish product 215, 245 can be processed for loading into a smoking device, such as a heat-not-burn device (as will be described later in this text).

Due to the manufacturing processes as described in FIGS. 1 and 2, the hybrid hashish products produced beneficially have cross sections that are substantially uniform along the length of the extruded elongated body. This uniformity is in shape, as well as in content of the hybrid hashish product, including the content of the cohesive mass and the distribution of the Cannabis plant biomass particles therein. For example, each hybrid hashish product in a batch of produced hybrid hashish products using the processes as described in FIGS. 1 and 2 can have a predictable cannabinoid content, e.g., of from about 5 wt. % to about 90 wt. %, including any value therein. The cannabinoid can be one or more of tetrahydrocannabinol (THC), cannabidiol (CBD), cannabinol (CBN), and any combinations thereof. Each hybrid hashish product can also have a predictable weight, e.g., from about 0.2 g to about 20 g, such as about 0.5 g, about 1.0 g, about 1.5 g, about 2.0 g, about 2.5 g, about 3.0 g, about 3.5 g, about 4.0 g, about 4.5 g, about 5.0 g, about 5.5 g, about 6.0 g, about 6.5 g, about 7.0 g, about 7.5 g, about 8.0 g, about 8.5 g, about 9.0 g, about 10.0 g, as about 10.5 g, about 11.0 g, about 11.5 g, about 12.0 g, about 12.5 g, about 13.0 g, about 13.5 g, about 14.0 g, about 14.5 g, about 15.0 g, about 15.5 g, about 16.0 g, about 16.5 g, about 17.0 g, about 17.5 g, about 18.0 g, about 18.5 g, about 19.0 g, or about 19.5 g, including any value therein. In some cases, a variability of about +/−10% on the measured weight can be acceptable to the manufacturer.

The Cannabis plant biomass particles can be substantially homogenously distributed throughout the cohesive mass and/or substantially homogenously distributed along the length of each hybrid hashish product. The Cannabis plant biomass particles can have a cannabinoid content of at least 5 wt. %. The Cannabis plant biomass particles can be from one or more strain(s) of Cannabis plant that are different from or the same as the one or more strain(s) of Cannabis plant of the isolated Cannabis trichomes.

In some embodiments, during the retrieving step 130, cutting step 160, or shaping step 170 of FIG. 2, notches can be formed along the length of the elongate body. The notches can be at regular intervals, such as between 40 mm and 120 mm, or any other suitable value. The notches can be shaped to denote specific quantities of hybrid hashish product. For example, each notched portion can represent a length of hybrid hashish product that easily is loaded in a tube or cone to obtain a smoking article. Each notched portion can be a contents indicator; for example, each notched portion can indicate a fixed weight, amount of THC or other cannabinoid content, etc. In addition to or instead of acting as markers, the notches can be configured to assist a user in removing the denoted portion of hybrid hashish product from the remaining shape.

The reader will understand that the process 100 or the process 150 may include a number of steps prior to or following exit of the substantially homogenous mixture from the mechanical mixing apparatus (e.g., the extruder). For example, a cooling step may be performed to cool down the substantially homogenous mixture to obtain a solid or semi-solid hybrid hashish product, either prior to passing through the die, after passing through the die, prior to cutting, after cutting, or any combination thereof.

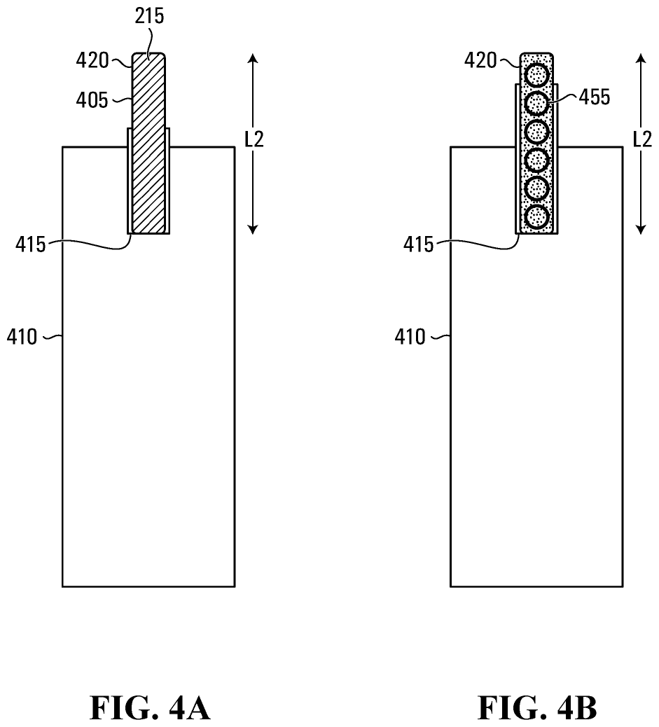

In some embodiments, as shown in FIG. 4, the elongated hybrid hashish product 215 (or elongated hybrid hashish product 245) can be loaded into into a device 410, e.g., a smoking device or a heat-not-burn device. Prior to use, the elongated hybrid hashish product 215 (or elongated hybrid hashish product 245) can be affixed to a mounting component 420 (either by the manufacturer or by the user). The mounting component 420 can be a mounting cartridge or tube that is loaded with the elongated hybrid hashish product 215 (or elongated hybrid hashish product 245) The elongated hybrid hashish product 215 (or elongated hybrid hashish product 245) together with the mounting component 420 forms a heatable elongated hybrid hashish product 405 that is suitable to be mounted to the heat-not-burn device 410. Each of the heatable elongated hybrid hashish products 405 can have a length L2. In some embodiments, the length L2 is configured so that the heatable elongated hybrid hashish product 405 sits within the mounting tube 415 within the heat-not-burn device 410. In some embodiments, the length L2 is configured to be approximately from about 40 mm to about 100 mm, such as 40 mm to about 90 mm, or from about 40 mm to about 80 mm, or from about 40 mm to about 70 mm, or from about 40 mm to about 60 mm, or about 50 mm (e.g., 2 inches). In some configurations, the heatable elongated hybrid hashish products 405 are ready-to-use products.

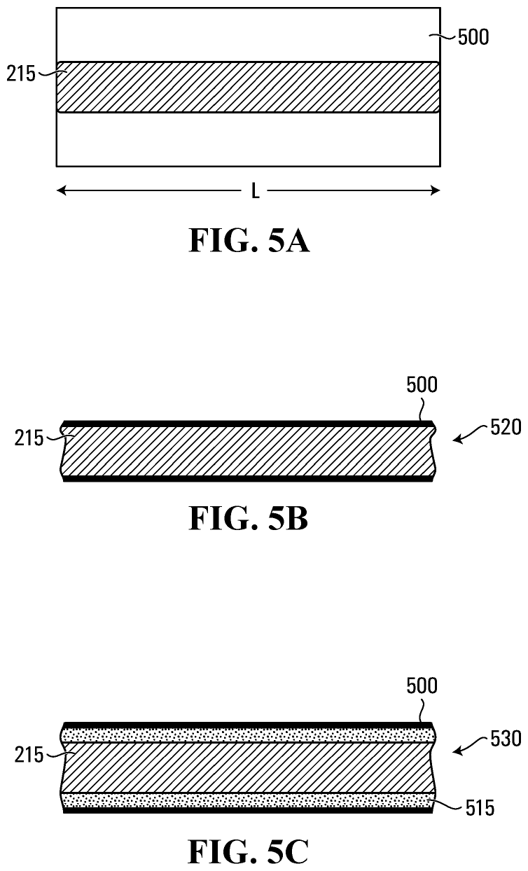

FIG. 5A illustrates an embodiment of the hybrid hashish product of the present disclosure that is configured to be loaded into a rolling medium or wrapper to form a smoking article. An elongated hybrid hashish product such as elongated hybrid hashish product 215 from FIG. 3 having a length L is placed on a rolling medium 500. The rolling medium or wrapper 500 may be any suitable rolling medium or wrapper known in the art, as discussed above. In this embodiment, the elongated hybrid hashish product 215 may have a length L that is configured to be approximately that one of a standard Cannabis cigarette length or equivalent to the length of the rolling medium 500. For example, the length L can be from about 40 mm to about 300 mm, or from 40 mm to about 120 mm. In FIG. 5B, the rolling medium 500 has been wrapped around the elongated hybrid hashish product 215 and the smoking article 520 is ready to use by a user. In FIB. 5C, the elongated hybrid hashish product 215 was loaded into the rolling medium 500 along with other additives shown as a layer 515 (e.g., smokable Cannabis products, or tobacco leaves) to form a smoking article 530 that is ready to use by a user.

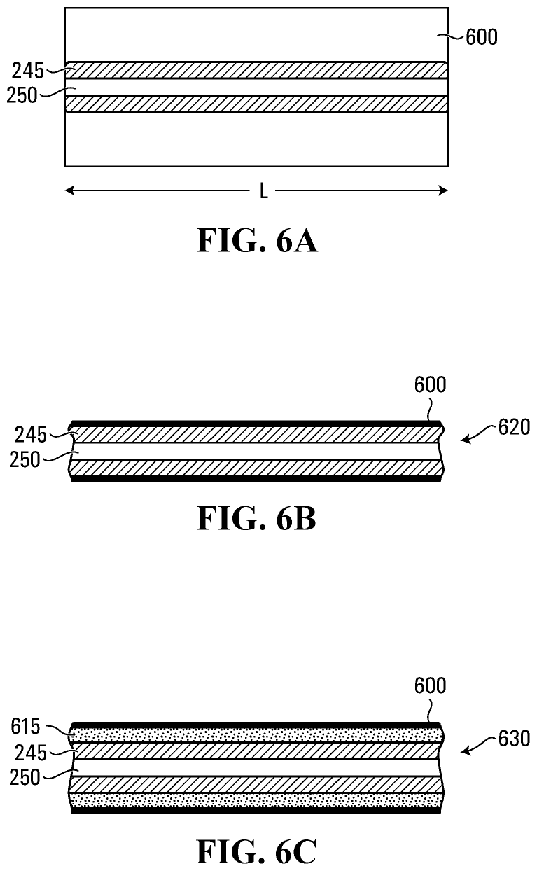

FIG. 6A illustrates an embodiment of the hybrid hashish product of the present disclosure that is configured to be loaded into a rolling medium or wrapper to form a smoking article with a hollow core. An elongated hybrid hashish product such as elongated hybrid hashish product 245 from FIG. 3 having a length L and a hollow core 250 (e.g., a hybrid hashish product with an annular cross-section) is loaded into a rolling medium 600. The rolling medium or wrapper 600 may be any suitable rolling medium or wrapper known in the art, as discussed above. In this embodiment, the elongated hybrid hashish product 245 may have a length L that is configured to be approximately that one of a standard Cannabis cigarette length or equivalent to the length of the rolling medium 600, as discussed above. In FIG. 6B, the rolling medium 600 has been wrapped around the elongated hybrid hashish product 245 to form smoking article 620, which is ready to use by a user. In FIG. 6C, the elongated hybrid hashish product 245 was loaded into the rolling medium 600 along with one or more additives shown as a layer 615 (e.g., smokable Cannabis products, or tobacco leaves) to form a smoking article 630 that is ready to use by a user. In the embodiments shown, the elongated hybrid hashish products 245 have a hollow core 250 that is the full length of the elongated hybrid hashish product 245, with a hollow diameter that is approximately 50% of the diameter of the entire hybrid hashish product, although other arrangements are also possible. The hollow core 250 may allow for more airflow, more even and smooth burn rate and a much smoother user experience when drawing on the hybrid hashish cigarette 620 or 630.

The reader will understand that the process 100 may include several steps prior to or following the steps shown. For example, a cooling step may be performed to cool down the cohesive mass to obtain a solid or semi-solid hybrid hashish product, either prior to passing through the die, after passing through the die, prior to cutting, after cutting, or any combination thereof.

The present disclosure thus describes methods that make it possible to produce hybrid hashish products at a high rate and at a lower cost. In addition, the hybrid hashish products thus produced may be designed to have different shapes while retaining a homogeneous distribution of its constituents, which was not possible with the previous methods (e.g., hand rolling or pressing). Further, the hybrid hashish products having elongated bodies that are described herein can provide a more pleasant user experience when handling same, since the hybrid product includes a combination of characteristics from Cannabis plant biomass and from isolated trichomes (hashish).

### Distribution Test

The detectable marker (e.g., Cannabis plant biomass particles and/or detectable molecule (e.g., cannabinoid)) distribution into the hybrid hashish product can be assessed using a distribution test. In this test, a hybrid hashish product is segmented along several axes using a cutting blade to obtain first (e.g., peripheral) and second (e.g. core) portions and the detectable marker content is determined thereafter. Note that for the purpose of the present description, this test procedure will be referred to as a “distribution test”.

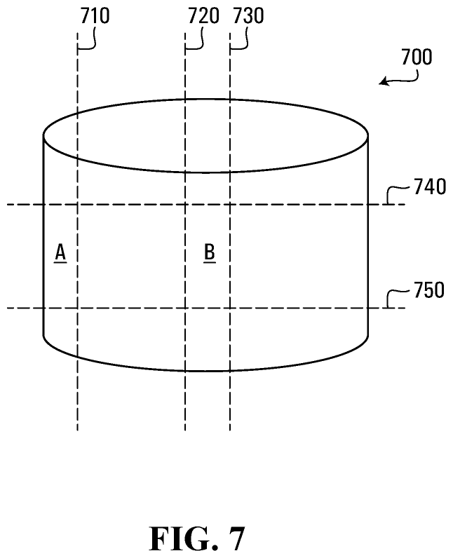

The test procedure is as follows:
A predetermined number of hybrid hashish product samples to be simultaneously tested (herein referred as “test samples”), which are all made in a single batch or individually but in a sufficiently controlled environment such as to ensure a high degree of uniformity between the samples are provided.
If desired, the test samples are conditioned for 1 h at a temperature of 20° C. and at a humidity level of 40%.
Each test sample is tested by placing same on a support surface. For test samples that are not spherical, the test samples are placed on the support surface in an orientation such that the same side of the test samples will face up, if applicable. A single blade can then be used to slice the test sample to obtain at least two portions thereof.
   For example, in embodiments where the test sample is voluminous enough (e.g., may include 2 g or more of mass and/or may have a size of sufficient dimensions), the single blade can be used to slice test sample 700 along two 2 lines 720, 730 along a longitudinal axis thereof, substantially parallel to each other, as shown in FIG. 7A. The single blade can then be used to slice the test sample 700 along two 2 lines 740, 750 along a transverse axis thereof, substantially parallel to each other. The single blade can then be used to slice the test sample 700 along 1 line 710 substantially parallel to lines 720, 730, and closer to the outer edge of test sample 700. The crossing of axes 720, 730 with 740, 750 produce a second portion B whereas the crossing of axes 740, 750 with 710 produces a first portion A. In this embodiment, while the first portion is shown as a peripheral portion and the second portion is shown as a core portion, the person of skill will readily understand that portions from other subsections of the test sample are also possible.
   For example, in embodiments where the test sample 700 is smaller in mass or size than the previous embodiment, the single blade can be used to slice the test sample 700 along a longitudinal line 760 to obtain a first portion A and a second portion B, as shown in FIG. 7B. Alternatively, the single blade can be used to slice the test sample 700 along a transverse line 770 to obtain a first portion A and a second portion B, as shown in FIG. 7C.
The detectable marker content of each of the first and second portions A, B is then determined, for example using visual inspection for the Cannabis biomass particles content or USP NF 731 Loss On Drying method to quantify water content, or any other suitable technique/equipment for any other detectable molecule (e.g., HPLC for a cannabinoid). The detectable marker content distribution is reported for each assay as per the following ratio B/A. A ratio R where R<0.90 or R>1.10 is considered a failure; in other words, variability of >10% in the detectable marker content between two portions of the test sample indicates a failure of the test.

The detectable marker content of various portions from the same test sample can be obtained as per variations of the above-described procedure in order to determine the detectable marker content at various location in the test sample and, thus, determine the detectable marker content distribution in the test sample.

The distribution of the detectable marker in the hybrid hashish product is substantially homogeneous, and the detectable marker can be detected in at least 90 vol. % of the hashish product. The levels (or contents) of the detectable marker in the hybrid hashish product is such that the first detectable marker content (e.g., in a core portion of the hashish product) and the second detectable marker content (e.g., in a peripheral portion of the hashish product) are present in a ratio first/second markers of from 0.85 to 1.15.

### Cohesiveness Test

The cohesiveness of a hashish product can be assessed using a cohesiveness test. In this test, a hybrid hashish product is shaped into a ball and submitted to the pressure of a predetermined weight for a predetermined period. The cohesiveness of the hashish product is qualitatively determined thereafter. Note that for the purpose of the present description, this test procedure will be referred to as a “cohesiveness test”.

The test procedure is as follows:
A predetermined number of hybrid hashish product samples to be simultaneously tested (herein referred as “test samples”) are provided.
If desired, the test samples are conditioned for 1 h at a temperature of 20° C. and at a humidity level of 40%.
Each test sample is formed into a ball, if possible and tested by placing same on a support surface.
A 10 kg weight is placed onto the test sample for 10 seconds and then removed.
Cohesiveness of the hashish product is determined based on the extent that the test sample crumbled under the 10 kg weight.

### EXAMPLES

The following example describes an exemplary mode of making and practicing certain compositions that are described herein. This example is for illustrative purposes only and are not meant to limit the scope of the compositions and methods described herein.

### Example 1

In this example, a hashish product was made using pre-treated isolated Cannabis trichomes that were mixed under conditions sufficient to obtain a cohesive mass.

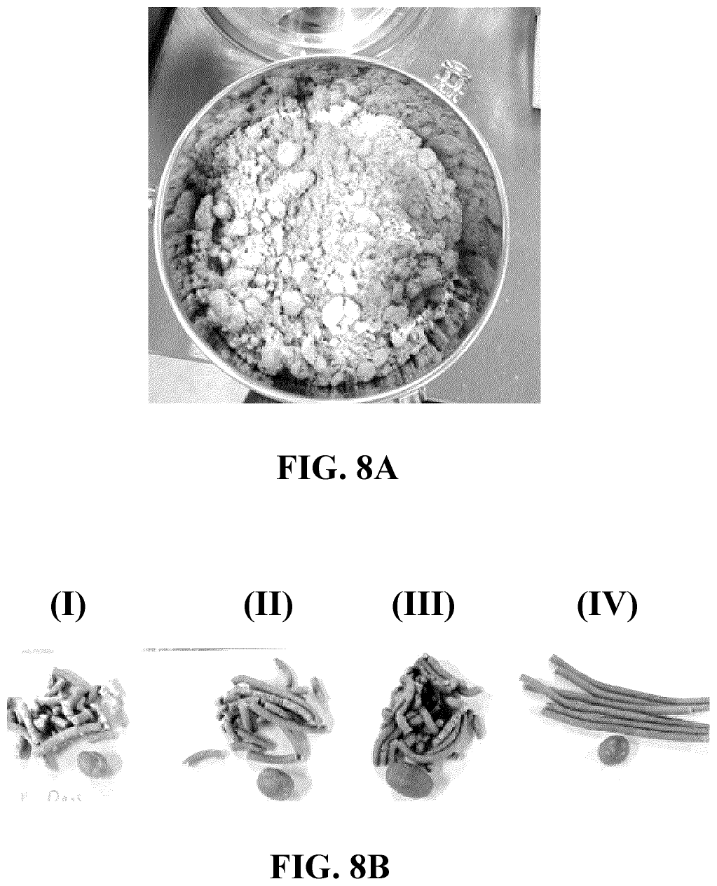

As for the pre-treatment, a batch of isolated Cannabis trichomes was pre-treated according to the conditions in Table 1. The pre-treated isolated Cannabis trichomes are shown in FIG. 8A.

TABLE 1
Pre-treatment process

| Input | Step 1 | Step 2 |
|---|---|---|
| Isolated cannabis trichomes | Pre-heating in oven at | Cooling down to |
| (164.4 g NL × BB strain) | 125° C. for 40 minutes | room temperature |
| sealed in container | | for 1 hour |

The pre-treated isolated Cannabis trichomes were then loaded into a food grinder model G1700 (GVode, Inc.) attached to Classic Series 4.5-Quart Tilt-Head Stand Mixer (KitchenAid, Canada) and retrieved from a round die having an aperture of 3 mm. The material was either mixed once, or the mixed material was re-loaded several times into the food grinder and reprocessed through the food grinder. Processing through the food grinder once is referred to herein as 1 pass, whereas processing 5 times is referred to herein as 5 passes.

Hashish products (BCO-007) obtained after 1 pass, 3 passes, 6 passes and 9 passes are shown in FIG. 8B. It was observed that the hashish product obtained after 6 passes had a shiny appearance that was consistent with the desired profile.

### Example 2

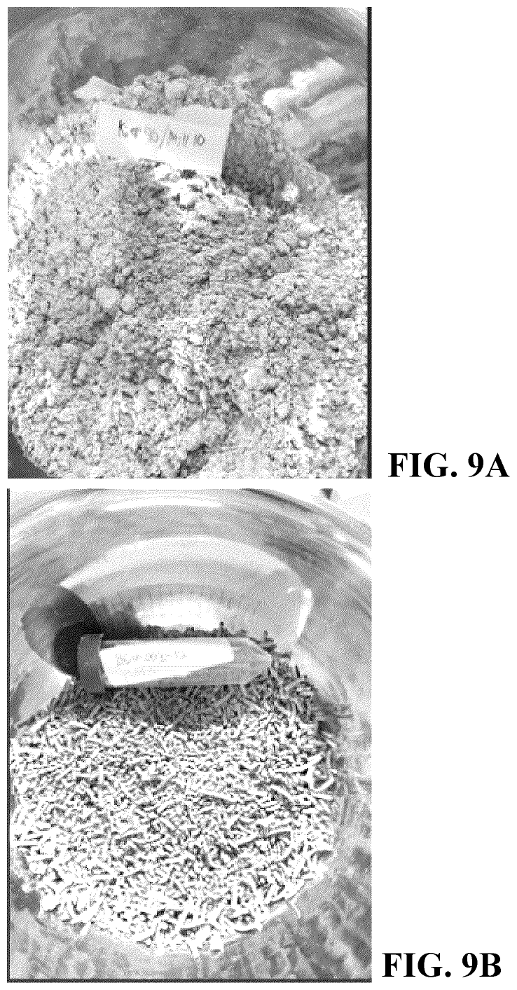

In this example, a hashish product was made using isolated Cannabis trichomes and milled Cannabis flower that were mixed under the conditions of Example 1 but failed to produce a cohesive mass. The mix of isolated Cannabis trichomes and milled Cannabis flower are shown in FIG. 9A.

A batch of 180 g of isolated Cannabis trichomes (NLxBB Cannabis strain) was mixed by hand with a batch of 20 g of milled Cannabis flowers (Grace strain) and then loaded into the food grinder attached to the head stand mixer.

Hashish products (BCU-001) were homogeneous and light colored after 1 pass but flaky—after 4 passes, as shown in FIG. 9B, the product was still homogenous in appearance, but did not form a cohesive mass as it crumbled to powder when pressed between fingers.

### Example 3

In this example, a hashish product was made using isolated Cannabis trichomes, milled Cannabis flower and water that were mixed under the conditions of Example 1 but failed to produce a cohesive mass.

A batch of 90 g of isolated Cannabis trichomes (NLxBB Cannabis strain) was mixed by hand with a batch of 10 g of milled Cannabis flowers and 15 g of water and then loaded into the food grinder attached to the head stand mixer.

Hashish products (BCU-002) obtained after 4 passes are shown in FIG. 10A. It was observed that the hashish products were homogeneous but did not form a cohesive mass as it crumbled to powder when pressed between fingers.

Next, the hashish products from the food grinder were pressed in mechanical press under conditions in Table 2 to result in a block of hashish as shown in FIG. 10B.

TABLE 2
Hashish pressing parameters

| Press | Mould size/ | Mass of | Press |
|---|---|---|---|
| load | geometry | pressed material | duration |
| 9400 lbs | 3 × 5/rectangular | 45 g | 5 min |

It was observed that the pressed hash was homogenous but still crumbled to powder when pressed between fingers.

### Example 4

In this example, a hashish product was made using pre-treated isolated Cannabis trichomes and pre-treated milled Cannabis flower that were mixed under the conditions of Example 1 and produced a cohesive mass.

As for the pre-treatment, a batch of isolated Cannabis trichomes was mixed with a batch of milled Cannabis flower and water in a container. The container was sealed, and the materials therein were pre-treated according to the conditions in Table 3.

TABLE 3
Pre-treatment process

| Input | Step 1 | Step 2 |
|---|---|---|
| Isolated cannabis trichomes | Pre-heating in oven at | Cooling down to |
| (90 g NL × BB strain), milled | 120° C. for 30 minutes | room temperature |
| cannabis flower (10 g) and | | |
| water (15 g) | | |

A mass loss of 2.5 g was observed (due to water evaporation) after pre-treatment while no release of resinous oily material was observed.

Hashish products (BCU-003) obtained after 5 passes had a cohesive and homogenous appearance.

### Example 5

In this example, a hashish product was made using pre-treated isolated Cannabis trichomes and pre-treated milled Cannabis flower that were mixed under the conditions of Example 1 and produced a cohesive mass.

As for the pre-treatment, a batch of isolated Cannabis trichomes were mixed with a batch of milled Cannabis flower and water in a container. The container was sealed, and the materials therein were pre-treated according to the conditions in Table 4.

TABLE 4
Pre-treatment process

| Input | Step 1 | Step 2 |
|---|---|---|
| Isolated cannabis trichomes | Pre-heating in oven at | Cooling down to |
| (80 g NL × BB strain), milled | 120° C. for 30 minutes | room temperature |
| cannabis flower (20 g) and | | |
| water (15 g) | | |

A mass loss of 2.2 g was observed (due to water evaporation) after pre-treatment. Hashish products (BCU-004) obtained after 1 pass are shown in FIG. 11A. It was observed that the hashish products obtained after 1 pass formed a cohesive mass.

Next, the hashish products from the food grinder were pressed in a mechanical press with the same conditions as outlined in Table 2 to result in a block of hashish as shown in FIG. 11B. It was observed that the pressed hash formed a cohesive mass.

### Example 6

In this example, a hashish product was made using pre-treated isolated Cannabis trichomes and pre-treated milled Cannabis flower that were mixed under the conditions of Example 1 and produced a cohesive mass.

As for the pre-treatment, a batch of isolated Cannabis trichomes was mixed with a batch of milled Cannabis flower and water in a container. The container was sealed, and the materials therein were pre-treated according to the conditions in Table 5.

TABLE 5
Pre-treatment process

| Input | Step 1 | Step 2 |
|---|---|---|
| Isolated cannabis trichomes | Pre-heating in oven at | Cooling down to |
| (70 g NL × BB strain), milled | 120° C. for 30 minutes | room temperature |
| cannabis flower (30 g) and | | |
| water (15 g) | | |

A mass loss of 1.8 g was observed (due to water evaporation) after pre-treatment. Hashish products (BCU-005) obtained after 1 pass formed a cohesive mass.

Next, the hashish product was pressed in a mechanical press with the same conditions as outlined in Table 2 to result in a block of hashish.

### Example 7

In this example, a hashish product was made using pre-treated isolated Cannabis trichomes and pre-treated milled Cannabis flower that were mixed under the conditions of Example 1 and produced a cohesive mass.

As for the pre-treatment, a batch of isolated Cannabis trichomes were mixed with a batch of milled Cannabis flower and water in a container. The container was sealed, and the materials therein were pre-treated according to the conditions in Table 6.

TABLE 6
Pre-treatment process

| Input | Step 1 | Step 2 |
|---|---|---|
| Isolated cannabis trichomes | Pre-heating in oven at | Cooling down to |
| (60 g NL × BB strain), milled | 120° C. for 30 minutes | room temperature |
| cannabis flower (40 g) and | | |
| water (15 g) | | |

A mass loss of 1.3 g was observed (due to water evaporation) after pre-treatment. Hashish products (BCU-006) obtained after 1 pass formed a cohesive mass.

Next, the hashish product was pressed in a mechanical press with the same conditions as outlined in Table 2 to result in a block of hashish (BCU-006) as shown in FIG. 14.

### Example 8

In this example, a hashish product was made using pre-treated isolated Cannabis trichomes that was mixed under the conditions of Example 1 and produced a cohesive mass.

As for the pre-treatment, a batch of isolated Cannabis trichomes was mixed with water in a container. The container was sealed, and the materials therein were pre-treated according to the conditions in Table 7.

TABLE 7
Pre-treatment process

| Input | Step 1 | Step 2 |
|---|---|---|
| Isolated cannabis trichomes | Pre-heating in oven at | Cooling down to |
| (100 g NL × BB strain) and | 120° C. for 30 minutes | room temperature |
| water (15 g) | | |

A mass loss of 1.7 g was observed (due to water evaporation) after pre-treatment. Hashish products (BCU-007) obtained after 1 pass was negatively affected by the addition of water as water was heated first during pre-treatment and lubrication caused by the water resulted in less shear in the grinder.

Next, the hashish product was pressed in a mechanical press with the same conditions as outlined in Table 2 to result in a block of hashish (BCU-006).

### Example 9

In this example, a hashish product was made using pre-treated isolated Cannabis trichomes and pre-treated milled Cannabis flower that were mixed under the conditions of Example 1 and produced a mass which was not cohesive.

As for the pre-treatment, a batch of isolated Cannabis trichomes was mixed with a batch of milled Cannabis flower and water in a container. The container was sealed, and the materials therein were pre-treated according to the conditions in Table 8.

TABLE 8
Pre-treatment process

| Input | Step 1 | Step 2 |
|---|---|---|
| Isolated cannabis trichomes | Pre-heating in oven at | Cooling down to |
| (50 g NL × BB strain), | 120° C. for 30 minutes | room temperature |
| cannabis milled flower (50 g) | | |
| and water (15 g) | | |

It was observed that mixer clogged while loading the mixture. A portion of the mixture (BCU-008) that passed the die (before the machine was clogged) did not show a cohesive mass and it crumbled to powder when pressed between fingers. These results suggest that with the strain used, the proportions of 50 g Cannabis plant biomass to 50 g of isolated trichomes does not produce a cohesive mass using a food grinder.

### Example 10

In this example, a hashish product was attempted to be made using pre-treated isolated Cannabis trichomes and pre-treated milled Cannabis flower that were mixed under the conditions of Example 1.

As for the pre-treatment, a batch of isolated Cannabis trichomes was mixed with water in a container. The container was sealed, and the materials therein were pre-treated according to the conditions in Table 9.

TABLE 9
Pre-treatment process

| Input | Step 1 | Step 2 |
|---|---|---|
| Isolated cannabis trichomes | Pre-heating in oven at | Cooling down to |
| (90 g NL × BB strain) | 120° C. for 30 minutes | room temperature |
| and water (10 g) | | |

It was observed that after 8 passes, the mixer clogged. This suggests that under these mixing conditions, 8 passes through the grinder does not provide a cohesive mass.

### Example 11

In this example, a hashish product was attempted to be made using pre-treated isolated Cannabis trichomes and pre-treated milled Cannabis flower that were mixed under the conditions of Example 1.

As for the pre-treatment, a batch of isolated Cannabis trichomes was mixed with water in a container. The container was sealed, and the materials therein were pre-treated according to the conditions in Table 10.

TABLE 10
Pre-treatment process

| Input | Step 1 | Step 2 |
|---|---|---|
| Isolated cannabis trichomes | Pre-heating in oven at | Cooling down to |
| (95 g NL × BB strain) | 120° C. for 30 minutes | room temperature |
| and water (5 g) | | |

It was observed that after 8 passes, the mixer clogged. This suggests that under these mixing conditions, 8 passes through the grinder does not provide a cohesive mass.

### Example 12

In this example, a hashish product was attempted to be made using pre-treated isolated Cannabis trichomes and pre-treated milled Cannabis flower that were mixed under the conditions of Example 1.

As for the pre-treatment, a batch of isolated Cannabis trichomes was placed in a container, the container was sealed, and the materials therein were pre-treated according to the conditions in Table 11.

TABLE 11
Pre-treatment process

| Input | Step 1 | Step 2 |
|---|---|---|
| Isolated cannabis trichomes | Pre-heating in oven at | Cooling down to |
| (100 g NL × BB strain) | 120° C. for 30 minutes | room temperature |

It was observed that after 8 passes, the mixer clogged. This suggests that under these mixing conditions, 8 passes through the grinder does not provide a cohesive mass.

### Example 13

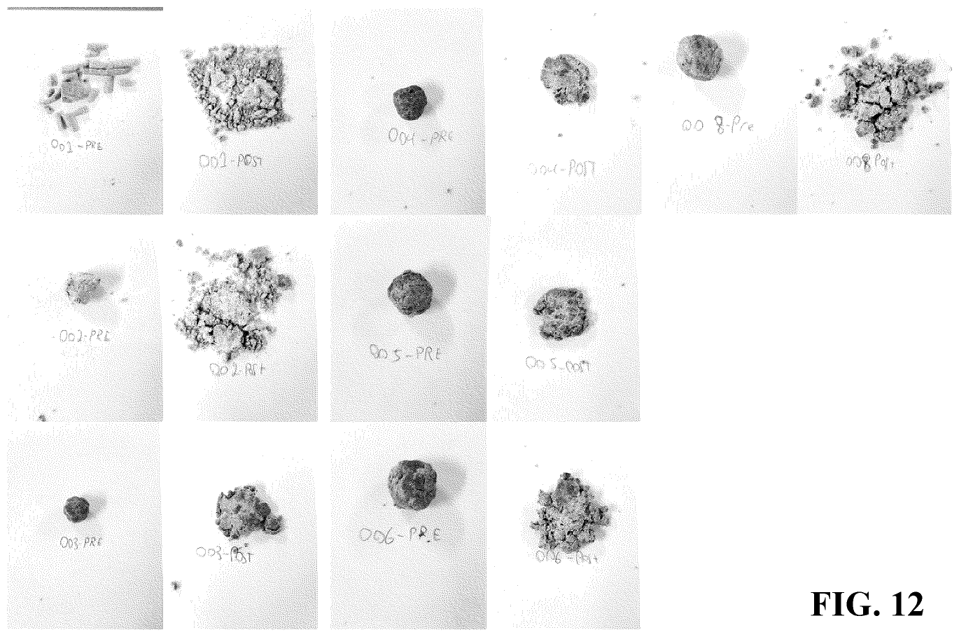

In this example, hashish products from the previous examples were submitted to the cohesiveness test. The results are shown in FIG. 12. The hashish identity is shown in handwriting where “001-PRE” indicates that it is a hashish sample from BCU-001 and before the cohesiveness test. The following table summarizes the hashish identification correlated to the example.

TABLE 12

| Hashish Identification | Example |
|---|---|
| 001 | 2 |
| 002 | 3 |
| 003 | 4 |
| 004 | 5 |
| 005 | 6 |
| 006 | 7 |
| 008 | 9 |

### OTHER EMBODIMENTS

In addition, although described primarily in the context of methods and systems, other implementations are also contemplated, as instructions stored on a non-transitory computer-readable medium, image processing, and/or control features for example.

Other examples of implementations will become apparent to the reader in view of the teachings of the present description and as such, will not be further described here.

Note that titles or subtitles may be used throughout the present disclosure for convenience of a reader, but in no way these should limit the scope of the invention. Moreover, certain theories may be proposed and disclosed herein; however, in no way they, whether they are right or wrong, should limit the scope of the invention so long as the invention is practiced according to the present disclosure without regard for any particular theory or scheme of action.

All references cited throughout the specification are hereby incorporated by reference in their entirety for all purposes.

Reference throughout the specification to “some embodiments”, and so forth, means that a particular element (e.g., feature, structure, and/or characteristic) described in connection with the invention is included in at least one embodiment described herein, and may or may not be present in other embodiments. In addition, it is to be understood that the described inventive features may be combined in any suitable manner in the various embodiments.

It will be understood by those of skill in the art that throughout the present specification, the term “a” used before a term encompasses embodiments containing one or more to what the term refers. It will also be understood by those of skill in the art that throughout the present specification, the term “comprising”, which is synonymous with “including,” “containing,” or “characterized by,” is inclusive or open-ended and does not exclude additional, un-recited elements or method steps.

Unless otherwise defined, all technical and scientific terms used herein have the same meaning as commonly understood by one of ordinary skill in the art to which this invention pertains. In the case of conflict, the present document, including definitions will control.

As used in the present disclosure, the terms “around”, “about” or “approximately” shall generally mean within the error margin generally accepted in the art. Hence, numerical quantities given herein generally include such error margin such that the terms “around”, “about” or “approximately” can be inferred if not expressly stated.

Although various embodiments of the disclosure have been described and illustrated, it will be apparent to those skilled in the art in light of the present description that numerous modifications and variations can be made. The scope of the invention is defined more particularly in the appended claims.

1. A hybrid hashish product, comprising a cohesive mass of agglomerated isolated Cannabis trichomes and particles of Cannabis plant biomass.

2. The hashish hybrid product according to claim 1, wherein the particles of Cannabis plant biomass are substantially homogeneously distributed throughout the cohesive mass.

3. The hybrid hashish product according to claim 1, wherein the particles of Cannabis plant biomass represent up to about 50 wt. %, preferably from about 10 wt. % to about 40 wt. %, the percentage being expressed relative to a total weight of the cohesive mass.

4. (canceled)

5. The hybrid hashish product according to claim 1, wherein the particles of the Cannabis plant biomass have a size that passes through a sieve having a 10.0 mm pore size or less, preferably a 2.0 mm pore size.

6. The hybrid hashish product according to claim 1, wherein the hybrid hashish product comprises a cannabinoid in an amount of from about 5 wt. % to about 90 wt. %, the percentage being expressed relative to a total weight of the cohesive mass.

7-8. (canceled)

9. The hybrid hashish product according to claim 1, further comprising one or more additional components, and wherein the one or more additional components comprises one or more cannabinoid(s), one or more terpene(s), one or more flavonoid(s), one or more flavoring agent(s), one or more coloring agent(s), or any combinations thereof.

10. The hybrid hashish product according to claim 9, wherein the one or more additional components is substantially homogenously distributed throughout the cohesive mass.

11-14. (canceled)

15. A method of making a hybrid hashish product, comprising
a) providing raw materials for making the hybrid hashish product, the raw materials comprising pre-treated isolated Cannabis trichomes and pre-treated Cannabis plant biomass, wherein the pre-treated isolated Cannabis trichomes and the pre-treated Cannabis plant biomass comprise a Cannabis oil layer on at least a portion of a respective surface thereof;
b) mixing the raw materials under conditions sufficient to obtain a cohesive mass of agglomerated isolated Cannabis trichomes and particles of Cannabis plant biomass.

16. The method according to claim 15, wherein providing the raw materials comprises preheating the isolated Cannabis trichomes and Cannabis plant biomass under conditions sufficient to ooze Cannabis oil out therefrom and obtain the Cannabis oil layer on the at least portion of the respective surface thereof.

17. The method according to claim 16, wherein the preheating is performed in presence of water.

18. The method according to claim 17, wherein the water is incorporated in an amount of up to about 15 wt. %, preferably from about 5 wt. % to about 15 wt. %, more preferably from about 10 wt. % to about 15 wt. %, the percentage being expressed relative to a total weight of the raw materials.

19. The method according to claim 16, wherein the preheating is performed in a sealed container.

20. The method according to claim 16, wherein the preheating is performed at a temperature of from about 70° C. to about 130° C., preferably from about 80° C. to about 125° C., more preferably from about 120° C. to about 125° C.

21. The method according to claim 16, wherein the preheating is performed for a duration of from about 10 minutes to about 60 minutes, preferably from about 30 minutes to about 40 minutes.

22. The method according to claim 15, wherein the pre-treated Cannabis plant biomass provided in the step a) is in the form of particles, wherein the particles have a size that passes through a sieve having a 10.0 mm pore size or less, preferably a 2.0 mm pore size.

23. The method according to claim 15, wherein the mixing causes pulverisation of the Cannabis plant biomass into particles, wherein the particles have a size that passes through a sieve having a 10.0 mm pore size or less, preferably a 2.0 mm pore size.

24. (canceled)

25. The method according to claim 15, wherein in step a), the Cannabis plant biomass being provided represents up to about 50 wt. %, preferably from about 10 wt. % to about 40 wt. %, the percentage being expressed relative to a total weight of the raw materials.

26. The method according to claim 15, wherein the hybrid hashish product comprises a cannabinoid in an amount of from about 5 wt. % to about 90 wt. %, the percentage being expressed relative to a total weight of the cohesive mass.

27-28. (canceled)

29. The method according to claim 15, wherein one or more additional components are incorporated into the hybrid hashish, and wherein the one or more additional components comprises one or more cannabinoid(s), one or more terpene(s), one or more flavonoid(s), one or more flavoring agent(s), one or more coloring agent(s), or any combinations thereof.

30-35. (canceled)

36. The method according to claim 15, wherein the mixing includes applying compression and shear forces to the raw materials via a plurality of interpenetrate helicoidal surfaces within an elongated enclosure.

37. (canceled)
